(19) United States
(12) Patent Application Publication
(10) Pub. No.: US 2022/0331286 A1
(43) Pub. Date: Oct. 20, 2022

(54) CANNABIS LIMITS CANCER STEM CELL GROWTH IN POORLY DIFFERENTIATED CANCERS

(71) Applicant: The Regents of the University of California, Oakland, CA (US)

(72) Inventor: Anahid JEWETT, Los Angeles, CA (US)

(21) Appl. No.: 17/707,134

(22) Filed: Mar. 29, 2022

Related U.S. Application Data
(60) Provisional application No. 63/176,581, filed on Apr. 19, 2021.

(51) Int. Cl. A61K 31/352; A61P 35/00

(52) U.S. Cl. CPC A61K 31/352; A61P 35/00

(57) ABSTRACT

The present invention provides a composition comprising a cannabinoid for inhibiting growth of cancer stem cells. This invention also provides methods of treating cancer comprising cancer stem cells by administering the said composition to patients in need thereof.

### CROSS-REFERENCE TO RELATED APPLICATIONS

This application claims the benefit of U.S. Provisional Application No. 63/176,581, filed on Apr. 19, 2021, the entire contents of which are incorporated herein in their entirety by this reference.

### BACKGROUND

Observations dating back to more than 50 years have evidenced similarities between cancer and embryonic development and that led to the hypothesis of the existence of cancer stem cells (CSCs). Stem cells are characterized by the capacity to self-renew and to generate differentiated progenies. The regulation of these processes is fundamental for the maintenance of the stem cell pool within a tissue. CSCs also features these processes mainly represented by tumor-initiating capacity, metastatic potential, and drug resistance. Recent growing evidences suggest that the tumor is composed of heterogeneous populations of cells with different levels of malignity and the tumor development is driven by a specialized cell subset, characterized by self-renewing, multi-potent, and tumor-initiating properties. These malignant cells are called CSCs and their maintenance is tightly ensured by the microenvironment and the stroma. They are probably generated from normal stem or precursor cells within tissues after mutations occur and are typically resistant to conventional treatments. This model has been studied and demonstrated especially for hematological diseases.

CSCs share common properties with normal stem cells and have multiple unique properties that maintain tumor growth and aggressiveness. A key feature of CSCs is their self-renewal capacity, which appears to be a driving force for initiating and maintaining tumorigenicity.

Self-renewal of CSCs can be maintained by several endogenous signaling pathways, such as Notch, Hedgehog, Wnt, B-cell-specific Moloney murine leukemia virus integration site 1 (Bmi1), Pten, Bmp, and TGF-β,64-70 which are frequently activated in human cancers.

Among these pathways, the roles of Notch and Bmi1 signaling in oral cancer stemness have been extensively documented. Activation of the Notch1 signaling pathway is critical for the maintenance of CSCs and requires binding of its ligands Jagged 1 (JAG1), JAG 2, and δ-like, followed by proteolytic release of the Notch intracellular domain (NICD) and activation of NICD downstream target genes.

A characteristic property of CSCs is their metastatic potential. Epithelial-mesenchymal transition (EMT) is known to confer migratory potential in cancer cells, and this process has crucial roles in cancer metastasis. EMT is a process by which epithelial cells lose their characteristics to gain the mesenchymal phenotype, thus leading to cell migration and invasion. During EMT, epithelium-specific protein expressions (e.g., cytokeratins and E-cadherin) are diminished, whereas expressions of mesenchymal-specific proteins (fibronectin, vimentin, and N-Cad) are elevated.

Oral/oropharyngeal squamous cell carcinoma (OSCC), is a common malignant tumor of the head and neck, and is currently the sixth most common cancer worldwide. In general, CSCs in oral squamous cell carcinoma (OSCC) can be isolated by either cell-surface markers or their unique functional properties. Nevertheless, no single marker and CSC property are capable of specifically isolating oral CSC populations from OSCC cells, suggesting the heterogeneity of CSC populations.

Pancreatic cancer is a lethal condition with poor outcomes and an increasing incidence. Pancreatic cancer is ranked as the 14th most common cancer and the 7th highest cause of cancer mortality in the world. Surgery offers the best possible cure for pancreatic cancer. However, 80% of pancreatic cancer patients are inoperable at diagnosis, and no curative treatment is available for advanced pancreatic cancer. Even after surgery, the 5-year survival rate for pancreatic cancer remains low (15-20%), with most patients dying because of metastatic disease and local recurrence. Cancer stem cells (CSCs), which are pluripotent, self-renewable, and capable of forming tumors, contribute to pancreatic cancer initiation and metastasis and are responsible for resistance to treatment.

Therefore, a critical need exists for developing therapeutically effective compositions and methods for treating cancers, especially treatments that help control or eliminate CSCs.

### SUMMARY OF THE INVENTION

The present invention is based, at least in part, on the discovery that cannabinoids described herein preferentially kill CSCs that drive malignancy in cancer, demonstrating their utility in treatment of cancer. CSCs are well known to be resistant to anti-cancer therapies (e.g., chemotherapy), thus it is surprising and unexpected that CSCs are particularly sensitive to cannabinoids of the present disclosure. Accordingly, these cannabinoids are useful in treating cancer, either alone or in combination with an anti-cancer therapy (e.g., chemotherapy, immunotherapy), where the cannabinoid and anti-cancer therapy (e.g., chemotherapy, immunotherapy) target both CSCs/undifferentiated cancer cells and well-differentiated cancer cells, respectively. Cannabinoids exert anti-cancer effects via cannabinoid receptors. Surprisingly, there is no difference in the copy number of the cannabinoid receptors on differentiated cancer cells vs. CSCs/undifferentiated/stem-like cancer cells, thereby uncoupling the number of receptors with the anti-cancer activity of the cannabinoids of the present disclosure.

In certain aspects, provided herein is a method of preventing or treating a cancer in a subject, comprising administering to the subject a composition comprising a cannabinoid or a pharmaceutically acceptable salt thereof.

In certain embodiments, the cancer comprises cancer stem cells, poorly differentiated cancer cells, and/or undifferentiated cancer cells.

In certain embodiments, the cancer comprises cancer cells with (a) an increased level of CD44, CD26, CD166, CD326, CD338, and/or CD133; (b) a decreased level of CD54, PD-L1, and/or MHC class I on the cancer cell surface compared to differentiated cells (e.g., differentiated cancer cells or differentiated non-cancerous cells) (e.g., preferably of the same cell type); and/or (c) susceptibility to NK cell-mediated cytotoxicity.

In certain embodiments, the subject is treated conjointly with at least one cancer therapy, optionally wherein the subject is treated with at least one cancer therapy before, after, or concurrently with the composition comprising a cannabinoid. In some such embodiments, the at least one cancer therapy is selected from a surgery, radiation therapy, chemotherapy, immunotherapy, or a combination thereof.

In some embodiments, the at least one cancer therapy is chemotherapy, optionally wherein the chemotherapy comprises CDDP.

In some embodiments, the at least one cancer therapy is immunotherapy. In some embodiments, the immunotherapy inhibits an immune checkpoint, e.g., an immune checkpoint selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In some embodiments, the immune checkpoint is PD-1 or PD-L1, preferably PD-1.

In some embodiments, the immunotherapy comprises an NK cell therapy.

In certain embodiments, the subject is treated with a composition comprising WIN 55,212-2, chemotherapy, and an immunotherapy, wherein the immunotherapy inhibits PD-1 or PD-L1, optionally wherein the subject is afflicted with a pancreatic cancer.

In certain embodiments, the composition comprising a cannabinoid is administered by inhalation, oral administration, parenteral administration, sublingual administration, topical administration, intravenous administration, intratumoral administration, intramuscular administration, or subcutaneous administration.

In certain embodiments, the method decreases the amount of at least one cell surface antigen on a cancer cell, wherein the at least one cell surface antigen is selected from CD44, CD26, CD166, CD326, CD338, CD133, CD54, MHC class I, and PD-L1.

In certain aspects, provided herein is a method of inhibiting the proliferation of a cancer cell, comprising contacting the cancer cell with a composition comprising a cannabinoid or a pharmaceutically acceptable salt thereof.

In some embodiments, the cancer cell is a cancer stem cell, a poorly differentiated cancer cell, and/or an undifferentiated cancer cell.

In some embodiments, the cancer cell has (a) an increased level of CD44, CD26, CD166, CD326, CD338, and/or CD133; (b) a decreased level of CD54, PD-L1, and/or MHC class Ion the cancer cell surface compared to differentiated cells (e.g., differentiated cancer cells or differentiated non-cancerous cells) (e.g., preferably of the same cell type); and/or (c) susceptibility to NK cell-mediated cytotoxicity.

In certain embodiments, the cancer cell is contacted conjointly with at least one cancer therapy, optionally wherein the cancer cell is contacted with at least one cancer therapy before, after, or concurrently with the composition comprising a cannabinoid.

In some embodiments, the at least one cancer therapy is selected from a surgery, radiation therapy, chemotherapy, immunotherapy, or a combination thereof.

In some embodiments, the at least one cancer therapy is chemotherapy, optionally wherein the chemotherapy comprises CDDP.

In some embodiments, the at least one cancer therapy is immunotherapy. In some embodiments, the immunotherapy inhibits immune checkpoint. In some embodiments, the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In some embodiments, the immune checkpoint is PD-1 or PD-L1, preferably PD-L1.

In some embodiments, the immunotherapy comprises an NK cell therapy.

In certain embodiments, the cancer cell is contacted with a composition comprising WIN 55,212-2, chemotherapy, and an immunotherapy, wherein the immunotherapy inhibits PD-1 or PD-L1, optionally wherein the cancer cell is a pancreatic cancer cell.

In certain embodiments, the cancer cell is contacted with the composition in vitro, ex vivo, or in vivo.

In certain embodiments, the method decreases the amount of at least one cell surface antigen on the cancer cell, wherein the at least one cell surface antigen is selected from CD44, CD26, CD166, CD326, CD338, CD133, CD54, MEW class I, and PD-L1.

In certain aspects, provided herein is a method of determining whether a subject afflicted with a cancer would benefit from a composition comprising a cannabinoid or a pharmaceutically acceptable salt thereof, the method comprising: a) determining the amount of at least one biomarker selected from CD44, CD26, CD166, CD326, CD338, CD133, CD54, PD-L1, and MEW class I in a subject sample; b) determining the amount of the at least one biomarker in a control (e.g., differentiated cells, e.g., differentiated cancer cells or differentiated non-cancerous cells) (e.g., preferably of the same cell type); and c) comparing the amount of the at least one biomarker detected in steps a) and b); wherein a significantly higher amount of CD44, CD26, CD166, CD326, CD338, and/or CD133; and/or a significantly lower amount of CD54, PD-L1, and/or MHC class I in the subject sample indicates that the subject would benefit from the composition comprising a cannabinoid or a pharmaceutically acceptable salt thereof.

In certain embodiments, the method further comprises recommending, prescribing, or administering a) the composition comprising the cannabinoid or a pharmaceutically acceptable salt thereof to the subject, if the subject is determined to benefit from the composition comprising a cannabinoid or a pharmaceutically acceptable salt thereof; or b) a therapy other than the composition comprising the cannabinoid or a pharmaceutically acceptable salt thereof to the subject, if the subject is determined not to benefit from the composition comprising a cannabinoid or a pharmaceutically acceptable salt thereof.

In certain aspects, provided herein is a method of identifying the likelihood of reducing proliferation of a cancer cell contacted with a composition comprising a cannabinoid or a pharmaceutically acceptable salt thereof, the method comprising: a) determining the amount of at least one biomarker selected from CD44, CD26, CD166, CD326, CD338, CD133, CD54, PD-L1, and MHC class I in a sample comprising a cancer cell; b) determining the amount of the at least one biomarker in a control (e.g., differentiated cells, e.g., differentiated cancer cells or differentiated non-cancerous cells) (e.g., preferably of the same cell type); and c) comparing the amount of the at least one biomarker detected in steps a) and b); wherein a significantly higher amount of CD44, CD26, CD166, CD326, CD338, and/or CD133; and/or a significantly lower amount of CD54, PD-L1, and/or MHC class I in the sample indicates that the composition comprising a cannabinoid or a pharmaceutically acceptable salt thereof would likely reduce proliferation of the cancer cell.

In certain embodiments, the method further comprises contacting the cancer cell with a) the composition comprising a cannabinoid or a pharmaceutically acceptable salt thereof, if the composition comprising a cannabinoid or a pharmaceutically acceptable salt thereof is determined to likely reduce proliferation of the cancer cell; or b) a therapy other than the composition comprising a cannabinoid or a pharmaceutically acceptable salt thereof, if the composition comprising a cannabinoid or a pharmaceutically acceptable salt thereof is determined to not likely reduce proliferation of the cancer cell.

Numerous embodiments are further provided that can be applied to any aspect of the present invention and/or combined with any other embodiment described herein. For example, in certain embodiments, the cannabinoid is a cannabinoid receptor agonist or antagonist.

In some embodiments, the cannabinoid is a cannabinoid receptor agonist, optionally wherein the cannabinoid is a cannabinoid receptor agonist of CB1R and/or CB2R.

In some embodiments, the cannabinoid is synthetic or naturally occurring.

In some embodiments, the composition comprising the cannabinoid is a pharmaceutical composition.

In some embodiments, the cannabinoid is WIN 55,212-2.

In some embodiments, the cancer or cancer cell is of a solid or a hematological cancer.

In some embodiments, the cancer or cancer cell is a metastatic cancer.

In some embodiments, the cancer or cancer cell is selected from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and glioblastoma.

In some embodiments, the cancer or cancer cell is selected from a pancreatic cancer and an oral cancer, optionally wherein the oral cancer is oral squamous carcinoma.

In certain embodiments, the subject is a mammal, e.g., a mouse or human.

### BRIEF DESCRIPTION OF THE DRAWINGS

FIG. 1A-1E show that OSCSCs are adversely affected more by WIN 55,212-2 than are OSCCs. Both OSCCs (oral squamous carcinoma cells) and OSCSCs (oral squamous carcinoma stem cells) were treated with different concentrations of WIN 55,212-2 (10, 25, 50, and 100 μM) or CDDP (50 μg/ml), and the cell numbers were determined after 24 hours of treatment.

FIG. 2 depicts the cytostatic effect of WIN 55,212-2. WIN 55,212-2, at low concentrations arrests cancer growth, and at higher concentrations kills the tumor.

FIG. 3A-3D show that OSCSCs are affected by WIN 55,212-2, but not CDDP. OSCSCs and OSCCs were treated with WIN 55,212-2 at 25, 50, 75 or 100 μM, or CDDP at 50 μg/ml for 24 hours.

FIG. 4A-4B show that both OSCCS and OSCSCs exhibit a decrease in MHC-1 expression when treated with WIN 55,212-2.

FIG. 5A-5D show that OSCCs and OSCSCs exhibit a decrease in the levels of surface receptor expression of CD44, CD54, B7H1 and MHC class I after 24-hour treatment with WIN 55,212-2.

FIG. 6A-6E show that PL12 tumors had a lower decrease in cell count when treated with WIN 55,212-2 when compared to MP2 tumors.

FIG. 7A-7F show that PL12 and MP2 pancreatic tumors exhibited lower levels of CD44, CD54, MHC class I, and B7H1 surface antigens expression after treatment with WIN 55,212-2 for 48 hours.

FIG. 8A-8B depict the stage of differentiation in pancreatic tumors correlated with susceptibility to NK cell-mediated cytotoxicity.

FIG. 9 shows that WIN 55,212-2 treatment induced more pronounced shift in morphology and decreased viability in stem-like OSCSCs more than in differentiated OSCCs. Tumor cells were cultured at 3×10⁵ cells per ml in 12 well plates and treated with different concentrations of WIN 55,212-2 as shown in the figure, and images of the cells were taken under 400× magnification using DMI6000 B inverted microscope and LAS X software. OSCCs and OSCSCs were treated with CDDP (50 μg/mL) and different concentrations of WIN 55,212-2 for 24 hours before the microscopic images were taken. Scale bar=50 μm.

FIG. 10A-10B show that WIN 55, 212-2 treatment induced a greater decrease in the growth of stem-like/poorly differentiated OSCSCs. Tumor cells were cultured at 3×10⁵ cells per ml in 12 well plates and treated with different concentrations of WIN 55,212-2 and CDDP as shown in the figure for 24 hours, and the detached cells were collected before the wells were washed with 1×PBS and the attached cells were harvested using trypsin-0.25% EDTA, and they were combined with detached cells and counted. The number of viable cells was counted under light microscope using Trypan Blue staining. One of three representative experiments is shown in (A). Compiled data from three independent experiments performed as in (A) is shown in (B). An unpaired, two tailed Student t test was performed for the statistical analysis using Prism-7 software to compare within treatment group. The following symbol represent the levels of statistical significance within each analysis, ** (p-value 0.001-0.01).

FIG. 11A-11D show that WIN 55,212-2 induced greater cell death in stem-like OSCSCs when compared to differentiated OSCCs. Tumor cells were cultured at 3×10⁵ cells per ml in 12 well plates and treated with different concentrations of WIN 55,212-2 and CDDP as shown in the figure for 24 hours, and the detached cells were collected before the wells were washed with 1×PBS and the attached cells were harvested using trypsin-0.25% EDTA, and they were combined with detached cells. Propidium iodide (PI) staining was used to determine the cell death by flow cytometry. One of four representative experiments is shown in (A) and (B), whereas compiled data from four independent experiments performed as in (A) and (B) are shown in (C). The compiled data of paired OSCCs and OSCSCs at the same concentration of WIN 55,212-2 treatment from four experiments are shown in (D). An unpaired two-tailed Student t test (C) or a paired two-tailed Student t test (D) was performed for the statistical analysis using Prism-7 software. The following symbol represent the levels of statistical significance within each analysis, * (p-value 0.01-0.05).

FIG. 12A-12E show that WIN 55,212-2 decreased the expression of CD44, CD54, B7H1 in both OSCCs and OSCSCs, but MHC class I was decreased only in OSCCs, likely due to decreased or lack of MHC class I expression on OSCSCs. OSCCs and OSCSCs were treated with WIN 55,212-2 (5-50 μM) for 24 hours and the levels of surface expression for CD44 (A), CD54 (B), B7H1(C) and MHC class I (D) were determined after antibody staining followed by flow cytometric analysis. One representative experiment is shown in (A-D). Three biological replicates compiled are shown in (E).

FIG. 13 shows that significant decrease in MHC class I expression was seen on OSCCs after treatment with WIN 55,212-2. Tumor cells were cultured at 3×10⁵ cells per ml in 12 well plates and treated with different concentrations of WIN 55,212-2 as shown in the figure, and the detached cells were collected before the wells were washed with 1×PBS and the attached cells were harvested with trypsin-0.25% EDTA, and they were combined with detached cells before they were stained with the PE conjugated antibodies to CD44, CD54, MHC class-I and PD-L1. Attune NxT flow cytometer were used to assess stained samples and the results were analyzed using FlowJo vX software. The results of two independent experiments are shown across a number of different concentrations of WIN 55,212-2. Data are shown as Mean±SD. An unpaired, two tailed Student t test was performed for the statistical analysis using Prism-7 software to compare within tested cell lines. The following symbol represent the levels of statistical significance within each analysis, * (p-value 0.01-0.05).

FIG. 14 shows that WIN 55,212-2 treatment induced more pronounced shift in morphology and decreased viability in stem-like MP2 tumor cells more than in well-differentiated PL-12 tumor cells. Tumor cells were cultured at 3×10⁵ cells per ml in 12 well plates and treated with different concentrations of WIN 55,212-2 as shown in the figure, and images of the cells were taken under 400× magnification using DMI6000 B inverted microscope and LAS X software. MP2 and PL-12 tumor cells were treated with CDDP (50 μg/mL) and different concentrations of WIN 55,212-2 for 24 hours before the microscopic images were taken. Scale bar=50 μm.

FIG. 15A-15B show that WIN 55,212-2 caused a greater decrease in cell numbers of stem-like MP2 when compared to PL-12. Tumor cells were cultured at 3×10⁵ cells per ml in 12 well plates and treated with different concentrations of WIN 55,212-2 and CDDP as shown in the figure for 24-48 hours, and the detached cells were collected before the wells were washed with 1×PBS and the attached cells were harvested using trypsin-0.25% EDTA, and they were combined with detached cells and counted. The number of viable cells was counted under light microscope using Trypan Blue staining. One of three representative experiments is shown in (A). Compiled data from three experiments performed as in (A) is shown in (B). An unpaired, two tailed Student t test was performed for the statistical analysis using Prism-7 software. The following symbol represent the levels of statistical significance within each analysis, * (p-value 0.01-0.05).

FIG. 16A-16D show that WIN 55,212-2 induced higher cell death in stem-like MP2 cells compared to differentiated PL-12, whereas the effects of CDDP were pronounced on both PL-12 and MP2. Tumor cells were cultured at 3×10⁵ cells per ml in 12 well plates and treated with different concentrations of WIN 55,212-2 and CDDP as shown in the figure for 24-48 hours, and the detached cells were collected before the wells were washed with 1×PBS and the attached cells were harvested using trypsin-0.25% EDTA, and they were combined with detached cells. PI staining was used to determine the cell death by flow cytometry. One of two representative experiments is shown in (A) and (B), whereas compiled data from two experiments is shown in (C) and the compiled data of paired MP2 and PL-12 at the same concentration of WIN 55,212-2 treatment is shown in (D). An unpaired two-tailed Student t test (C) or a paired two-tailed Student t test (D) was performed for the statistical analysis using Prism-7 software. The following symbols represent the levels of statistical significance within each analysis, * (p-value 0.01-0.05), ** (p-value 0.001-0.01).

FIG. 17A-17F show that expression of CD44, CD54 and MHC class I was decreased in MP2 and PL12 cells, while B7H1 was increased in PL12 and decreased in MP2 after WIN 55, 212-2 treatment. PL12 and MP2 pancreatic tumor cells were treated with WIN 55,212-2 for 48 hours and the levels of surface markers expression for CD44 (A), CD54 (B), MHC class I (C) and B7H1 (D) were determined after antibody staining followed by flow cytometric analysis. One representative experiment is shown in (A-D). Three biological replicates compiled are shown in (E). (F) Expression of CD44, CD54 and MHC class I was decreased in MP2 and PL-12 tumor cells, while PD-L1 was increased in PL-12 and decreased in MP2 after WIN 55, 212-2 treatment. Tumor cells were cultured at 3×10⁵ cells per ml in 12 well plates and treated with different concentrations of WIN 55,212-2 as shown in the figure, and the detached cells were collected before the wells were washed with 1×PBS, and the attached cells were harvested with trypsin-0.25% EDTA, and they were combined with detached cells before they were stained with the PE conjugated antibodies to CD44, CD54, MHC class I and PD-L1. Attune NxT flow cytometer were used to run the samples and the results were analyzed using FlowJo vX software. The results of two independent experiments are shown across a number of different concentrations of WIN 55,212-2. Data are shown as Mean±SD. An unpaired, two tailed Student t test was performed for the statistical analysis using Prism-7 software to compare within tested cell lines. The following symbols represent the levels of statistical significance within each analysis, * (p-value 0.01-0.05), ** (p-value 0.001-0.01).

FIG. 18A-18D show a representative experiment of surface receptor expression of well-differentiated and poorly differentiated/stem like oral tumor cells. OSCCs and OSCSCs were treated with WIN 55,212-2 for 24 hours and the levels of surface receptor expression for CD44 (A), CD54 (B), MHC class I (C), and PDL1 (D) were determined by Attune Nxt flow cytometer and the results were analyzed using FlowJo vX software.

FIG. 19A-19D show a representative experiment of surface receptor expression of well-differentiated and poorly differentiated/stem like pancreatic tumor cells. PL-12 and MP2 pancreatic tumors were treated with WIN 55, 212-2 for 48 hours and the levels of surface receptor expression for CD44 (A), CD54 (B), MHC class I (C) and B7H1 (D) were determined by Attune Nxt flow cytometer and the results were analyzed using FlowJo vX software.

FIG. 20A-20B show that expression of CB2R but not CB1R on differentiated and stem-like/poorly differentiated tumor cells with differentiated tumors having higher expression of CB2R. Tumor cells were cultured at 3×10⁵ cells per ml in 12 well plates before the wells were washed with 1×PBS and the tumors were harvested with trypsin-0.25% EDTA before they were stained with the PE conjugated antibodies to CB1R and CD44 and Alexa fluor 488 conjugated CB2R (shown in red). Isotypec control antibodies were used to exclude non-specific staining (shown in blue). Attune NxT flow cytometer were used to run the samples and the results were analyzed using FlowJo vX software. One of two independent experiments is shown in (A). OSCSCs were cultured at 3×10⁵ cells per ml in 12 well plates before they were treated with the combination of IFN-γ (long/ml) and TNF-α (long/ml) for 48 hours. The tumors were then washed with 1×PBS and detached by the use of trypsin-0.25% EDTA before they were stained with Alexa fluor 488 conjugated CB2R antibodies. The stained sample were assessed using Attune NxT flow cytometer, and the results were analyzed using FlowJo vX software (B).

FIG. 21 shows that WIN 55,212-2 treatment induced more pronounced shift in morphology in stem like OSCSCs when compared to well-differentiated OSCCs. Tumor cells were cultured at 3×10⁵ cells per ml in 12 well plates and treated with different concentrations of WIN 55,212-2 as shown in the figure, and images of the cells were taken under 400× magnification using DMI6000 B inverted microscope and LAS X software. OSCCs and OSCSCs were treated with CDDP (50 μg/mL) and different concentrations of WIN 55,212-2 for 24 hours before the microscopic images were taken.

FIG. 22A-22C show that WIN 55,212-2 induced greater cell death in stem-like OSCSCs when compared to differentiated OSCCs. No differences between untreated tumor cells and those treated with the vehicle alone (DMSO) in cell count or the amount of cell death in OSCCs and OSCSCs. Tumor cells were cultured at 3×10⁵ cells/ml in 12 well plates and either left untreated or treated with the highest concentrations of DMSO used to solubilize WIN 55,212-2 for 24 hours, and the detached cells were collected before the wells were washed with 1×PBS and the attached cells were harvested using trypsin-0.25% EDTA, and they were combined with detached cells and analyzed by flow cytometry to determine the proportion of the live cells that had lost Forward and side scatter indicating decreased viability of the cells. In addition, the numbers of viable cells were determined after PI staining followed by flow cytometric analysis (FIG. 22A). OSCC tumor cells were treated as in FIG. 22A and the cells were analyzed by flow cytometry to determine the proportion of the live cells that had lost Forward and side scatter indicating decreased viability of the cells (FIG. 22B). In addition, the numbers of viable OSCC tumor cells were determined after PI staining followed by flow cytometric analysis (FIG. 22B). Tumor cells were cultured at 3×10⁵ cells per ml in 12 well plates and treated with different concentrations of WIN 55,212-2 and CDDP as shown in the figure for 24 hours, and the detached cells were collected before the wells were washed with 1×PBS and the attached cells were harvested using trypsin-0.25% EDTA, and they were combined with detached cells. PI staining was used to determine the cell death by flow cytometry. One of four representative experiment is shown in (FIG. 22B). The compiled data of paired OSCCs and OSCSCs at the same concentration of WIN 55,212-2 treatment from four experiments is shown in (FIG. 22C). A paired two-tailed Student t test was performed for the statistical analysis using Prism-7 software. The following symbol represent the levels of statistical significance within each analysis, * (p-value 0.01-0.05).

### DETAILED DESCRIPTION OF THE INVENTION

The present invention may be understood more readily by reference to the following detailed description which forms a part of this disclosure. It is to be understood that this invention is not limited to the specific methods, products, conditions, or parameters described and/or shown herein, and that the terminology used herein is for the purpose of describing particular embodiments by way of example only and is not intended to be limiting of the claimed invention.

### Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent to perform its intended function. Examples of routes of administration for treatment of a body which can be used include injection (subcutaneous, intravenous, parenteral, intraperitoneal, intratumoral, intrathecal, etc.), oral, inhalation, and transdermal routes. The injections can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

In some embodiments, the cannabinoid agent can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp. 197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The term “altered copy number” refers to increased or decreased copy number (e.g., germline and/or somatic) of a biomarker DNA as compared to the copy number of the biomarker DNA in a control sample. The term “altered amount” of a biomarker includes an increased or decreased RNA level or protein level of a biomarker in a sample, e.g., a cancer sample, as compared to the corresponding protein level in a normal, control sample. Similarly, the term “altered activity” of a biomarker includes an increased or decreased activity of the biomarker protein in a sample as compared to the corresponding activity in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors. An altered amount or activity of a biomarker protein may be determined by detecting posttranslational modification such as phosphorylation status of the marker, which may affect the expression or activity of the biomarker protein. An altered amount or activity of a biomarker protein may be due to a differentiation state of a cancer cell. An altered amount or activity of a biomarker protein may also be due to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to the expression products in the test sample. Such a control may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In some embodiments, the control may comprise differentiated cancer cells, CSCs, or heterogeneous cancer cells at various stages of differentiation. In other embodiments, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In some embodiments, the control may comprise normal or non-cancerous cell/tissue sample. In other embodiments, the control may comprise an expression level for a set of patients, such as a set of cancer patients, or for a set of cancer patients receiving a certain treatment, or for a set of patients with one outcome versus another outcome. In other preferred embodiments, the control may comprise normal cells, cells from patients treated with combination chemotherapy, and cells from patients having benign cancer. In still other embodiments, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In other embodiments, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample.

The amount of a biomarker in a cell is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the cell can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The term “subject” or “patient” refers to any healthy or diseased animal, e.g., any human or non-human animal. The non-human animal can be a vertebrate, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

In some embodiments, the subject is afflicted with cancer. In various embodiments, the subject is in need of and/or benefit from the compositions and methods of the present disclosure. In various embodiments of the methods of the present invention, the subject has not undergone treatment, such as chemotherapy, radiation therapy, targeted therapy, and/or immunotherapies. In other embodiments, the subject has undergone treatment, such as chemotherapy, radiation therapy, targeted therapy, and/or immunotherapies. In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

A “therapeutically effective amount” of a substance or cells is an amount capable of producing a medically desirable result in a treated patient, e.g., decrease tumor burden, decrease the growth of tumor cells, or alleviate any symptom associated with cancer, with an acceptable benefit: risk ratio, preferably in a human or non-human mammal.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

In some of the experiments described herein, MFI (mean fluorescence intensity) is used to compare expression targets of interest (TOI) across samples/cell populations using flow cytometry. MFI gives reliable information about expression/presence of TOI within the experiment. PE is an abbreviation for R-phycoerythrin, which is a fluorescent red dye. PI (propidium iodide) binds to DNA, but since it does not permeate cell walls it only detects DNA from dead cells.

### Cannabinoid Compositions

In certain aspects, the present disclosure provides a composition comprising a cannabinoid (e.g., cannabinoid agonist) for a treatment of cancer. In some embodiments, the cancer comprises a cancer stem cell. In certain embodiments, the composition further comprises the cannabinoid agonist which is provided in an amount of between about 0.01 and 1000 mg for dosing. In some embodiments, the administration of the composition is selected from inhalation, parenteral administration, oral administration, sublingual administration, and topical administration. In some embodiments, the cannabinoid is WIN 55,212-2. In certain embodiments the composition also comprises a DNA-interacting agent, an antimetabolite, a tubulin-interacting agent, a molecular-targeted therapeutic agent, an epigenetic-action inhibitor, a hormone and/or another cannabinoid.

In some embodiments, the composition is used to treat a cancer stem cell. In some embodiments, the cancer stem cell is a squamous carcinoma stem cell, multiple myeloma stem cell, melanoma stem cell, prostate cancer stem cell, ovarian cancer stem cell, oral cancer stem cell, colon cancer stem cell, pancreatic cancer stem cell, brain tumor stem cell. In certain embodiments, said squamous carcinoma stem cell is oral squamous carcinoma stem cell.

In certain embodiments, said treatment causes the cancer to enter a state of static growth. In some embodiments, said treatment causes cancer cell death. In certain embodiments, the cell death is autophagic, apoptotic, or necrotic.

In certain aspects, the present invention provides a method of treating a cancer stem cell comprising administering to a subject in need thereof an effective amount of a composition comprising a cannabinoid or a pharmaceutically acceptable salt thereof. In some embodiments, the cannabinoid is WIN 55,212-2. In certain embodiments the composition also comprises a DNA-interacting agent, antimetabolite, tubulin-interacting agent, molecular-targeted therapeutic agent, epigenetic-action inhibitor, hormone and/or another cannabinoid.

In some embodiments, the method is to treat a cancer stem cell. In some embodiments, the cancer stem cell is a squamous carcinoma stem cell, multiple myeloma stem cell, melanoma stem cell, prostate cancer stem cell, ovarian cancer stem cell, oral cancer stem cell, colon cancer stem cell, pancreatic cancer stem cell, brain tumor stem cell. In certain embodiments, said squamous carcinoma stem cell is an oral squamous carcinoma stem cell.

In certain aspects, the present invention provides a composition for a treatment of a poorly differentiated cancer comprising a cannabinoid agonist as an effective component. In certain embodiments, the composition further comprises the cannabinoid agonist which is provided in an amount of between about 0.01 and 1000 mg for dosing. In some embodiments, the administration of the composition is selected from inhalation, parenteral administration, oral administration, sublingual administration, and topical administration. In some embodiments, the cannabinoid is WIN 55,212-2. In certain embodiments the composition also comprises a DNA-interacting agent, an antimetabolite, a tubulin-interacting agent, a molecular-targeted therapeutic agent, an epigenetic-action inhibitor, a hormone and/or another cannabinoid.

In some embodiments, the composition is used to treat a poorly differentiated cancer. In some embodiments, the poorly differentiated cancer comprises a squamous carcinoma stem cell, multiple myeloma stem cell, melanoma stem cell, prostate cancer stem cell, ovarian cancer stem cell, oral cancer stem cell, colon cancer stem cell, pancreatic cancer stem cell, brain tumor stem cell. In certain embodiments, said squamous carcinoma stem cell is oral squamous carcinoma stem cell.

In certain embodiments, said treatment causes the cancer to enter a state of static growth. In some embodiments, said treatment causes cancer cell death. In certain embodiments, the cell death is autophagic, apoptotic, or necrotic.

In certain aspects, the present invention provides a method of treating a poorly differentiated cancer comprising administering to a subject in need thereof an effective amount of a composition comprising a cannabinoid or a pharmaceutically acceptable salt thereof. In some embodiments, the cannabinoid is WIN 55,212-2. In certain embodiments the composition also comprises a DNA-interacting agent, antimetabolite, tubulin-interacting agent, molecular-targeted therapeutic agent, epigenetic-action inhibitor, hormone and/or another cannabinoid.

In some embodiments, the method is to treat a poorly differentiated cancer. In some embodiments, the poorly differentiated cancer comprises a squamous carcinoma stem cell, multiple myeloma stem cell, melanoma stem cell, prostate cancer stem cell, ovarian cancer stem cell, oral cancer stem cell, colon cancer stem cell, pancreatic cancer stem cell, brain tumor stem cell. In certain embodiments, said squamous carcinoma stem cell is an oral squamous carcinoma stem cell.

In certain aspects, the present invention provides a composition for a treatment of an undifferentiated cancer comprising a cannabinoid agonist as an effective component. In certain embodiments, the composition further comprises the cannabinoid agonist which is provided in an amount of between about 0.01 and 1000 mg for dosing. In some embodiments, the administration of the composition is selected from inhalation, parenteral administration, oral administration, sublingual administration, and topical administration. In some embodiments, the cannabinoid is WIN 55,212-2. In certain embodiments the composition also comprises a DNA-interacting agent, an antimetabolite, a tubulin-interacting agent, a molecular-targeted therapeutic agent, an epigenetic-action inhibitor, a hormone and/or another cannabinoid.

In some embodiments, the composition is used to treat an undifferentiated cancer. In some embodiments, the undifferentiated cancer comprises a squamous carcinoma stem cell, multiple myeloma stem cell, melanoma stem cell, prostate cancer stem cell, ovarian cancer stem cell, oral cancer stem cell, colon cancer stem cell, pancreatic cancer stem cell, brain tumor stem cell. In certain embodiments, said squamous carcinoma stem cell is oral squamous carcinoma stem cell.

In certain embodiments, said treatment causes the cancer to enter a state of static growth. In some embodiments, said treatment causes cancer cell death. In certain embodiments, the cell death is autophagic, apoptotic, or necrotic.

In certain aspects, the present invention provides a method of treating an undifferentiated cancer comprising administering to a subject in need thereof an effective amount of a composition comprising a cannabinoid or a pharmaceutically acceptable salt thereof. In some embodiments, the cannabinoid is WIN 55,212-2. In certain embodiments the composition also comprises a DNA-interacting agent, antimetabolite, tubulin-interacting agent, molecular-targeted therapeutic agent, epigenetic-action inhibitor, hormone and/or another cannabinoid.

In some embodiments, the method is to treat a poorly differentiated cancer. In some embodiments, the poorly differentiated cancer comprises a squamous carcinoma stem-like cell, multiple myeloma stem-like cell, melanoma stem-like cell, prostate cancer stem-like cell, ovarian cancer stem-like cell, oral cancer stem-like cell, colon cancer stem-like cell, pancreatic cancer stem-like cell, brain tumor stem-like cell. In certain embodiments, said squamous carcinoma stem-like cell is an oral squamous carcinoma stem-like cell.

In any of the embodiments herein, the composition or method comprises a cannabinoid agonist. Non-limiting examples of cannabinoid agonists include, CP-55,940, WIN 55,212-2, JWH-015, JWH-133, SR141716 (rimonabant), SR144528, and ACEA. CP 55,940 is a cannabinoid which mimics the effects of naturally occurring tetrahydrocannabinol (THC) (a cannabinoid). The molecular weight is 376.6, and the its chemical name is (−)-cis-3-[2-Hydroxy-4-(1,1-dimethylheptyl)phenyl]-trans-4-(3-hydroxypropyl)cyclohexanol.

WIN 55,212-2 is a chemical described as an aminoalkylindole derivative, which produces effects similar to those of cannabinoids such as THC but has an entirely different chemical structure. The molecular weight is 426.5, and its chemical name is (R)-(+)-[2,3-Dihydro-5-methyl-3-(4-morpholinylmethyl)pyrrolo[1,2,3-de]-1,4-benzoxazin-6-yl]-1-naphthalenylmethanone mesylate.

JWH-015 is a chemical from the naphthoylindole family that acts as a subtype-selective cannabinoid agonist. The molecular weight is 327.4, and its chemical name is (2-methyl-1-propyl-1H-indol-3-yl)-1-naphthalenyl-methanone

JWH 133 is a synthetic cannabinoid (CB) that is a subtype-selective cannabinoid agonist. Its molecular weight is 312.5, and its chemical name is 3-(1,1-dimethylbutyl)-6aR,7,10,10aR-tetrahydro-6,6,9-trimethyl-6H-dibenzo[b,d]pyran.

SR141716 (rimonabant) is an anorectic antiobesity drug that is a subtype-selective cannabinoid inverse agonist. Its molecular weight is 463.8, and its chemical name is 5-(4-chlorophenyl)-1-(2,4-dichlorophenyl)-4-methyl-N-1-piperidinyl-1H-pyrazole-3-carboxamide.

SR144528 is a drug that acts as a potent and highly subtype-selective cannabinoid inverse agonist. The molecular weight is 476 and its chemical structure is 5-(4-chloro-3-methylphenyl)-1-[(4-methylphenyl)methyl]-N-[(1S,2S,4R)-1,3,3-trimethylbicyclo[2.2.1]hept-2-yl]-1H-pyrazole-3-carboxamide

Arachidonyl-2′-chloroethylamide (ACEA) is a synthetic subtype-specific cannabinoid agonist. Its molecular weight is 366, and its chemical structure is N-(2-chloroethyl)-5Z,8Z,11Z,14Z-eicosatetraenamide.

In some embodiments, administration of the composition is selected from inhalation, oral administration, parenteral administration, sublingual administration, and topical administration.

The dosage of the cannabinoid agonist, or a derivative thereof, administered to a patient may vary and may be an amount of from about 0.2 mg/kg to about 50 mg/kg, based on the weight of the patient. Thus, the dosage of the cannabinoid, or a derivative thereof, may vary depending upon, inter alia, nature of the disorder, the sex of the patient, i.e. male or female, etc. and may be about 0.2-50 mg/kg, about 1-45 mg/kg, about 10-40 mg/kg, about 20-40 mg/kg, about 25-35 mg/kg, based on the weight of the patient. The dosage of the cannabinoid, or a derivative thereof, may vary depending upon, inter alia, the severity of the disorder, the nature of the disorder, the sex of the patient, i.e. male or female, etc. and may be about 1 μmol (about 2.3 mg in the case of WIN55,212-2), about 10 μmol (about 23 mg), about 20 μmol (about 47 mg), about 30 μmol (about 70 mg), about 40 μmol (about 93 mg), about 45 μmol (about 105 mg), about 50 μmol (about 117 mg), about 55 μmol (about 129 mg), about 60 μmol (about 141 mg), about 65 μmol (about 152 mg), about 70 μmol (about 164 mg), about 75 μmol (about 176 mg), about 80 μmol (about 187 mg), about 85 μmol (about 200 mg), about 90 μmol (about 211 mg), about 95 μmol (about 223 mg), or about 100 μmol (about 234 mg). Other cannabinoids may be provided at the corresponding amounts.

The effective amounts of compound or drug can and will vary according to the specific composition being utilized, the mode of administration and the age, weight and condition of the subject. Dosages for a particular individual subject can be determined by one of ordinary skill in the art using conventional considerations. In general, the amount of cannabinoid agent will be between about 0.01 to about 1000 milligrams per day and more typically, between about 0.5 to about 750 milligrams per day and even more typically, between about 1.0 to about 500 milligrams per day, between about 1.0 to about 100 milligrams per day, between about 5.0 to about 100 milligrams per day, and between about 20.0 to about 100 milligrams per day. The daily dose can be administered in one, two, three or four doses per day.

It will be understood by the person skilled in the art that the dosage regimen and the frequency of administration may be tailored depending upon, inter alia, the severity of the disorder, the nature of the disorder, the sex of the patient, i.e., male or female, etc. and may be for example, generally based on a dose regime of once weekly, twice weekly, three times weekly, four times weekly, five times weekly, six times weekly, or every day; for one week in a 3-week cycle. Alternatively, the dosage regime may be generally based on a dose regime of once weekly, twice weekly, three times weekly, four times weekly, five times weekly, six times weekly, or every day; for two weeks in a 3-week cycle. Alternatively, the dosage regime may be generally based on a dose regime of once weekly, twice weekly, three times weekly, four times weekly, five times weekly, six times weekly, or every day; for 3 weeks in a 3-week cycle. Alternatively, the dosage regime may be generally based on a dose regime of once weekly, twice weekly, three times weekly, four times weekly, five times weekly, six times weekly, or every day; for one week in a 4-week cycle. Alternatively, the dosage regime may be generally based on a dose regime of once weekly, twice weekly, three times weekly, four times weekly, five times weekly, six times weekly, or every day; for two weeks in a 4-week cycle. Alternatively, the dosage regime may be generally based on a dose regime of once weekly, twice weekly, three times weekly, four times weekly, five times weekly, six times weekly, or every day; for 3 weeks in a 4-week cycle. Alternatively, the dosage regime may be generally based on a dose regime of once weekly, twice weekly, three times weekly, four times weekly, five times weekly, six times weekly, or every day; for 4 weeks in a 4-week cycle.

When the cannabinoid agonist, or a derivative thereof, is administered by way of infusion, the duration of the infusion may vary. Thus, the infusion may be administered as an intravenous infusion over a period of 15 minutes, 30 minutes, 45 minutes, 1 hour, 1.5 hours, 2 hours, 2.5 hours, 3 hours, 3.5 hours, 4 hours, 4.5 hours, 5 hours, 5.5 hours, or 6 hours, each treatment day during a cycle. The dosing may be once a day. The dosing can also be multiple times a day. The dose can be q.d. (once a day), t.i.d. (three times a day), q.i.d. (four times a day), q4h, q3h, q2h, and q1h.

The dose can be over the lifetime of the patient. The dose can also be continued until symptoms resolve. The dose can be continued until the cancer is no longer seen by biopsy or other relevant diagnostic measures. The dose regime can be altered throughout the lifetime of the dosing of the patient. It can be altered if the cancer stops growing. It may also be tapered off to zero or a maintenance dose if the cancer has gone into remission, stopped growing, or otherwise become benign.

### Pharmaceutical Compositions

Generally speaking, the pharmacokinetics of the particular agent to be administered will dictate the most preferred method of administration and dosing regimen. The cannabinoid agent can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Moreover, the cannabinoid agent can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavoring agent such as peppermint, oil of Wintergreen, or cherry flavoring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound(s) may be incorporated into sustained-release preparations and formulations, including those that allow specific delivery of the active peptide to specific regions of the gut.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Pharmaceutically acceptable carriers and/or diluents include any and all solvents, dispersion media, coatings, antibacterial and antifungal agents, isotonic and absorption delaying agents and the like.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the active ingredients may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier. Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension.

In the case of a spray, this may be achieved for example by means of a metering atomizing spray pump. To improve nasal delivery and retention the compounds according to the invention may be encapsulated with cyclodextrins or formulated with other agents expected to enhance delivery and retention in the nasal mucosa.

Administration to the respiratory tract may also be achieved by means of an aerosol formulation in which the active ingredient is provided in a pressurized pack with a suitable propellant such as a chlorofluorocarbon (CFC) for example dichlorodifluoromethane, trichlorofluoromethane, or dichlorotetrafluoroethane, carbon dioxide, or other suitable gas.

The aerosol may conveniently also contain a surfactant such as lecithin. The dose of drug may be controlled by provision of a metered valve.

Alternatively, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier may form a gel in the nasal cavity. The powder composition may be presented in unit dose form for example in capsules or cartridges of, e.g. gelatin, or blister packs from which the powder may be administered by means of an inhaler.

Additional embodiments of pharmaceutical compositions are provided below. As used herein the pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

A pharmaceutical composition of the present invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds including, e.g., cannabinoids may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, cannabinoids are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present invention are dictated by, and directly dependent on, the unique characteristics of the active compound, the particular therapeutic effect to be achieved, and the limitations inherent in the art of compounding such an active compound for the treatment of individuals.

### Methods of Detection

In certain aspects, provided herein is a method of detecting at least one biomarker. In some embodiments, a biomarker is differentially expressed in CSCs, undifferentiated, or partially differentiated cancer cells, when compared with normal cells or differentiated cancer cells. For example, CD44 is highly expressed in CSCs compared with differentiated cancer cells. Additional stem cell markers that show higher expression on CSCs include CD26, CD166, CD326, CD338, and CD133. By contrast, CD54, PD-L1, and/or MHC Class I molecule are highly expressed in differentiated cancer cells compared with CSCs. Such biomarkers are useful in determining whether a subject would benefit from the treatment with certain cannabinoids of the present disclosure.

In other embodiments, a biomarker is differentially expressed in cancer cells after treatment with cannabinoids of the present disclosure. Representative biomarkers are described in working Examples, and detection of such biomarker(s) allow determining the efficacy of the cannabinoids and/or prognosis of a subject treated with the cannabinoids.

### Detecting Biomarker Expression and Amount

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In some embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In other embodiments, detecting or determining expression levels of a biomarker and functionally similar homologs thereof, including a fragment or genetic alteration thereof (e.g., in regulatory or promoter regions thereof) comprises detecting or determining RNA levels for the marker of interest. In some embodiments, one or more cells from the subject to be tested are obtained and RNA is isolated from the cells. In some embodiments, a sample of tissue cells is obtained from the subject.

In some embodiments, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in preferred embodiments, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In some embodiments, poly(A)+RNA is isolated from the RNA sample. In general, such purification takes advantage of the poly-A tails on mRNA. In particular, and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In certain preferred embodiments, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope encompassed by the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos: 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In some embodiments, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In some embodiments, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In other embodiments, hybridization under “stringent conditions” occurs when there is at least 97% identity between the sequences.

The form of labeling of the probes may be any that is appropriate, such as the use of radioisotopes, for example, 32P and 35S. Labeling with radioisotopes may be achieved, whether the probe is synthesized chemically or biologically, by the use of suitably labeled bases.

In certain embodiments, the biological sample contains polypeptide molecules from the test subject. Alternatively, the biological sample can contain mRNA molecules from the test subject or genomic DNA molecules from the test subject.

In other embodiments, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

### Methods for Detection of Biomarker Amount or Activity

The activity or level of a biomarker protein can be detected and/or quantified by detecting or quantifying the expressed polypeptide. The polypeptide can be detected and quantified by any of a number of means well-known to those of skill in the art. Decreased levels of polypeptide expression of the polypeptides encoded by a biomarker nucleic acid and functionally similar homologs thereof, including a fragment or genetic alteration thereof (e.g., in regulatory or promoter regions thereof) are associated with the de-differentiation of cells (e.g., cancer cells). Any method known in the art for detecting polypeptides can be used. Such methods include, but are not limited to, immunodiffusion, immunoelectrophoresis, radioimmunoassay (MA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and MA procedures may be conducted such that a desired biomarker protein standard is labeled (with a radioisotope such as 125I or 35S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In some embodiments, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzymes are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including 125I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (125I, 121I), carbon (14C), sulphur (35S), tritium (3H), indium (112In), and technetium (99mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a Kd of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art. As described above, antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

### Methods for Detection of Biomarker Structural Alterations

The following illustrative methods can be used to identify the presence of a structural alteration in a biomarker nucleic acid and/or biomarker polypeptide molecule in order to, for example, identify one or more biomarkers described herein.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

### Exemplary Diseases

When the use herein described comprises the treatment of cancer, the cancer may be selected from one or more of primary cancer, breast cancer, colon cancer, prostate cancer, non-small cell lung cancer, glioblastoma, lymphoma, melanoma, mesothelioma, liver cancer, intrahepatic bile duct cancer, oesophageal cancer, pancreatic cancer, stomach cancer, laryngeal cancer, brain cancer, ovarian cancer, testicular cancer, cervical cancer, oral cancer, pharyngeal cancer, renal cancer, thyroid cancer, uterine cancer, urinary bladder cancer, hepatocellular carcinoma, thyroid carcinoma, osteosarcoma, small cell lung cancer, leukaemia, myeloma, gastric carcinoma and metastatic cancers.

Other uses can include other diseases in which treatment includes targeting of poorly differentiated, undifferentiated, stem cell-like, or stem cells as opposed to the differentiated cells. Examples of these indications include bone marrow transplant and graft-vs-host disease.

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, lei omyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

### Combination Therapy

In some embodiments, a cannabinoid of the present disclosure is administered conjointly with an additional therapy. In some embodiments, the additional therapy is a cancer therapy. In some embodiments, the pharmaceutical composition further comprises an additional therapy (e.g., cancer therapy) other than a cannabinoid of the present disclosure. Any suitable additional therapy may be used provided that the activity of the additional therapy and/or the cannabinoid is not grossly diminished when combined. In other embodiments, an additional therapy is not part of the pharmaceutical composition comprising a cannabinoid but is nonetheless administered conjointly to a subject.

The therapeutic agents of the present invention can be used alone or can be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents of the present invention can be administered with a therapeutically effective dose of chemotherapeutic agent.

In other embodiments, agents of the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Suitable anti-cancer drugs include trastuzumab or protein tyrosine kinase inhibitors (e.g. lapatinib). In some embodiments, the subject has previously been administered, or is currently being administered, an aromatase inhibitor. In some embodiments, the aromatase inhibitor is selected from aminoglutethimide, testolactone, anastrozole, letrozole, exemestane, vorozole, formestane, megestrol acetate, and fadrozole. In some embodiments the anti-cancer drug is a hormone agonist or antagonist. In some embodiments the hormone antagonist or hormone agonist is an ER antagonist. Non-limiting exemplary ER antagonists include tamoxifen and fulvestrant or a combination thereof.

In some embodiments, the cancer therapy is a selective estrogen receptor modulator. Selective estrogen receptor modulators are a class of medicines that act upon the estrogen receptor. Their action is different in various tissues, thereby granting the possibility to selectively inhibit or stimulate estrogen-like action in various tissues. Selective estrogen receptor modulators include: afimoxifene (4-hydroxytamoxifen), arzoxifene, bazedoxifene, clomifene, lasofoxifene, ormeloxifene, ormeloxifene, raloxifene, tamoxifen, or toremifene and they are used for a variety of medical indications.

Some selective estrogen receptor modulators used as anti-tumoral agents include raloxifene, tamoxifen, or toremifine.

In alternative embodiments, the cancer therapy may be an alkylating agent. An alkylating agent is a type of anti-neoplastic agent that works by interfering with DNA in several ways. Alkyl groups, are added to DNA, which causes the cell to degrade the DNA as the cell tries to replace them. Alkylating agents also interfere with the bonds between DNA strands, preventing the DNA from separating, which is a step required in DNA replication. Also, the alkylating agents can create mismatching DNA-base pairs by converting one DNA base into a different one.

All these changes occur when a cell is preparing to divide, and the permanent damage they cause results in cessation of division and cell death.

Preferably the alkylating agent is selected from the group consisting of: alkyl sulfonates, busulfan, ethyleneimines and methylmelamines, hexamethymelamine, altretamine, thiotepa, nitrogen mustards, cyclophosphamide, mechlorethamine, mustine, uramustine, uracil mustard, melphalan, chlorambucil, ifosfamide, nitrosureas, carmustine, cisplatin, streptozocin, triazenes, dacarbazine, imidazotetrazines, and temozolomide. Alkylating agents used as anti-tumoral agents include cisplatin, temozolamide, and carmustine.

Antimetabolites are only similar to normal metabolites found within the cell. When cells incorporate an antimetabolite into their cellular metabolism, the proper functioning of the cell is interfered with, usually preventing the cell from dividing. Antimetabolites interfere with specific phases of the cell-cycle. Antimetabolites are classified according to the substances with which they interfere, i.e., they antagonize or inhibit folic acid, pyrimidine, purine, and adenosine deaminase. Examples include: Folic acid antagonist: methotrexate; pyrimidine antagonists: 5-Fluorouracil, 5-fluorodeoxyuridine, cytosine arabinoside, capecitabine, and gemcitabine; purine antagonists: 6-Mercaptopurine and 6-Thioguanine; adenosine deaminase inhibitors: 2-chloro-2′-deoxyadenosine, fludarabine and pentostatin.

In any of the foregoing embodiments, the cannabinoid agonist and the one or more other agents among those described herein may be combined into a single dosage unit, or they may be administered in separate dosage units at the same time or at different times.

In some embodiments, the cancer therapy is an immunotherapy. Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer. Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the cancer vaccine is administered in combination with one or more inhibitors of immune checkpoints, such as PD1, PD-L1, and/or CD47 inhibitors.

Adoptive cell-based immunotherapies can be combined with the therapies of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C (indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like. Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein. Similarly, agents and therapies other than immunotherapy or in combination thereof can be used with in combination with an anti-KHK antibodies to treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In other embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA. In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

### Use of Cannabinoid Compositions

The present invention provides for methods of determining whether a subject would benefit from the compositions and methods provided herein, as well as the prophylactic and therapeutic methods of treating a subject at risk of (or susceptible to) a cancer. The cancer may be a solid or hematological cancer.

### Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, certain aspects encompassed by the present invention relates to diagnostic assays for determining the amount and/or activity level of a biomarker described herein in the context of a biological sample (e.g., cancer cells) to thereby determine whether an individual afflicted with a condition that would benefit from a composition comprising a cannabinoid (e.g., WIN 55,212-2). Such assays can be used for prognostic or predictive purpose alone, or can be coupled with a therapeutic intervention to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the figures, examples, and otherwise described in the specification; or one or more biomarkers known in the art (e.g., those biomarkers that identify CSCs or stem-like/undifferentiated cancer cells).

### Diagnostic Assays

The present invention provides, in part, methods, systems, and code for accurately classifying whether a biological sample is associated with a condition that would benefit from the compositions of the present disclosure. In some embodiments, the present invention is useful for classifying a sample (e.g., from a subject) as associated with or at risk for a condition that would benefit from the cannabinoid compositions, e.g., comprising cannabinoid(s) that preferentially kill CSCs or stem-like cancer cells.

An exemplary method for detecting the amount or activity of a biomarker described herein, and thus useful for classifying whether a sample is likely or unlikely to respond to a cannabinoid of the present disclosure (e.g., WIN 55,212-2) involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely cannabinoid responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method encompassed by the present invention further comprises sending the sample classification results to a clinician, e.g., an oncologist.

In some embodiments, the diagnosis of a subject is followed by administering to the individual a defined treatment based upon the diagnosis.

In some embodiments, the methods further involve obtaining a control biological sample (e.g., biological sample from a subject who does not have a condition that would benefit from a cannabinoid of the present disclosure (e.g., WIN 55,212-2)), a biological sample from the subject during remission, or a biological sample from the subject during treatment for developing a condition that would benefit from a cannabinoid of the present disclosure (e.g., WIN 55,212-2).

### Prophylactic Methods

In certain aspects, the present invention provides a method for preventing in a subject, a disease or condition associated with cancer. Subjects at risk for a disease that would benefit from treatment with the claimed agents or methods can be identified, for example, by any or a combination of diagnostic or prognostic assays known in the art. Administration of a prophylactic agent can occur prior to the manifestation of symptoms associated with cancer. The appropriate agent used for treatment (e.g. cannabinoids and/or cannabinoids in combination with a cancer therapy) can be determined based on clinical indications and can be identified.

### Therapeutic Methods

Another aspect encompassed by the present invention pertains to therapeutic methods of inhibiting the proliferation of a cancer cell by administering the compositions described herein. The therapeutic compositions described herein can be used in a variety of in vitro and in vivo therapeutic applications using the formulations and/or combinations described herein. In some embodiments, the therapeutic agents can be used to treat cancers determined to be responsive thereto. For example, single or combination therapy can be used to treat cancers in subjects identified as likely responders thereto.

Modulatory methods encompassed by the present invention involve contacting a cell, such as a cancer cell, with a composition comprising a cannabinoid described herein. Exemplary compositions useful in such methods are described above. Such compositions can be administered in vitro or ex vivo (e.g., by contacting the cell with the composition) or, alternatively, in vivo (e.g., by administering the agent to a subject). As such, the present invention provides methods useful for treating an individual afflicted with a condition that would benefit from the compositions described herein.

As described above, in certain instances, it may be desirable to further administer an additional therapy, e.g., cancer therapy. In certain embodiments, the method further comprises surgery, radiation therapy, chemotherapy, immunotherapy, or a combination thereof. In certain embodiments, the method further comprises immunotherapy which includes, NK-therapy, CAR-T therapy, and antibody therapy.

In certain embodiments, treatment with a compound or therapy described herein causes the cancer to enter a state of static growth. In some embodiments, said treatment causes cancer cell death. In certain embodiments, the cell death is autophagic, apoptotic, or necrotic.

### Clinical Efficacy

Clinical efficacy can be measured by any method known in the art. For example, the response to a therapy (e.g., a cannabinoid or a combination therapy provided herein), relates to e.g., any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al. (2007) J. Clin. Oncol. 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer vaccine therapeutic regimen is at least 25%, 30%, 35%, 40%, 45%, 50%, 55%, 60%, 65%, 70%, 75%, 80%, 85%, or more.

Additional criteria for evaluating the response to a therapy (e.g., a cannabinoid or a combination therapy provided herein) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular agent encompassed by the present invention can be administered to a population of subjects and the outcome can be correlated to biomarker measurements that were determined prior to administration of a therapy (e.g., a cannabinoid or a combination therapy provided herein). The outcome measurement may be pathologic response to therapy given in the neoadjuvant setting. Alternatively, outcome measures, such as overall survival and disease-free survival can be monitored over a period of time for subjects following a therapy (e.g., a cannabinoid or a combination therapy provided herein). In certain embodiments, the same doses of the agent are administered to each subject. In related embodiments, the doses administered are standard doses known in the art for the agent. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

### Kits

The present invention also encompasses kits. For example, the kit can comprise a cannabinoid or a combination therapy of the present disclosure, and/or any agent that is useful in detecting a biomarker described herein, packaged in a suitable container and can further comprise instructions for using such reagents. The kit may also contain other components, such as administration tools packaged in a separate container.

### EXAMPLES

### Example 1: Effect of Synthetic Cannabinoids on Well Differentiated and Cancer Stem Cells

### Methods and Materials for Examples 2-5

### Cell Death Assays

Oral squamous carcinoma cells (OSCCs) and oral squamous carcinoma stem cells (OSCSCs) were isolated from cancer patients with tongue tumors at UCLA. The cells were cultured in RPMI 1640 complete medium with 10% fetal bovine serum (FBS) 1.4% Antibiotic-Antimycotic, 1% sodium pyruvate, 1.4% non-essential amino acids, 1% L-glutamine, 0.2% gentamicin (Gemini Bio-Products, CA, USA), and 0.15% sodium bicarbonate (Fisher Scientific, PA, USA).

### Surface Staining and Cell Death Assays

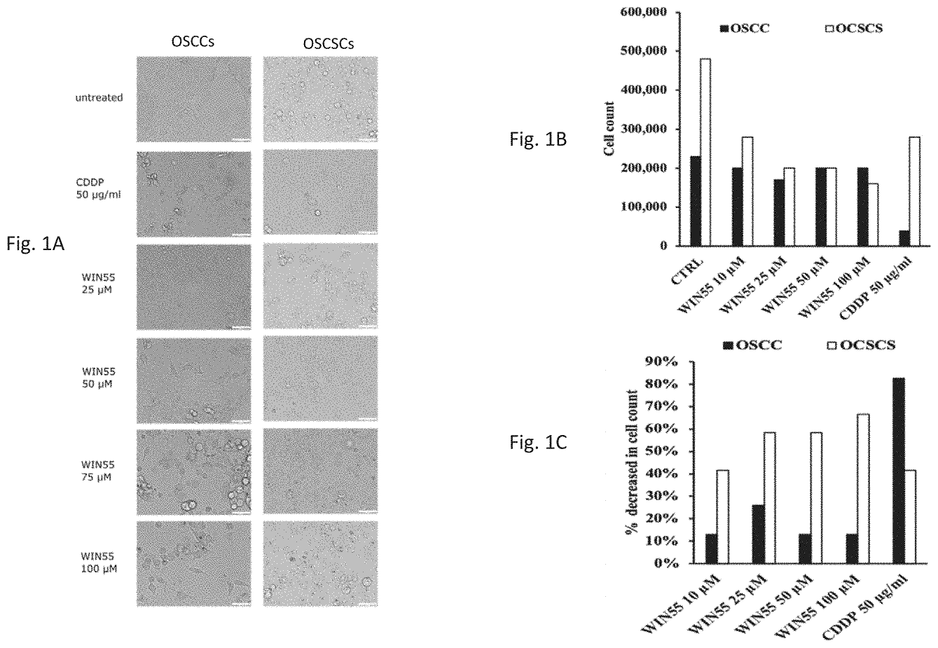

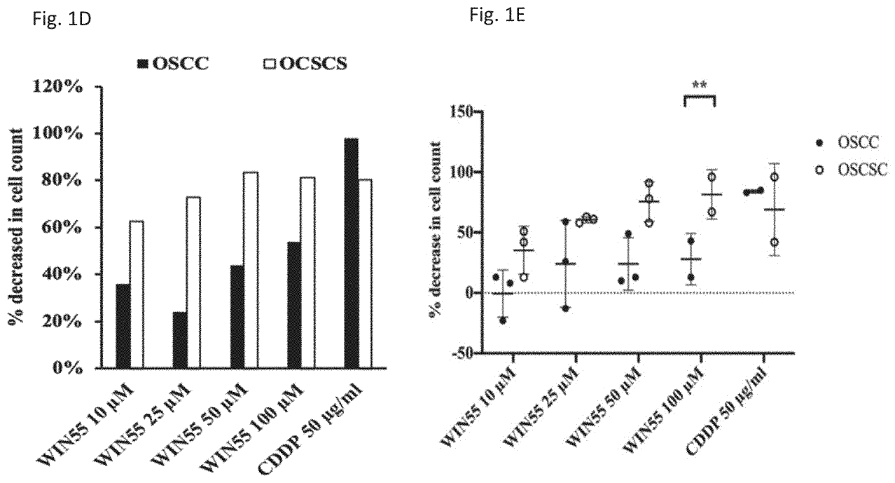

OSCCs and OSCSCs were treated with different concentrations of WIN 55,212-2 or cis-diaminedichloridoplatinum (II) (CDDP; cisplatin) as shown in FIGS. 1B, 1C, and 1E and cultured overnight at 37 C in 5% CO2. After an overnight incubation the cells were washed twice and stained with propidium iodide (PI) at a concentration of 3 μg/ml. Flow cytometric analysis was performed using Attune flow cytometer results were analyzed using FlowJo vX software (Ashland, Oreg.).

Briefly, cells were analyzed based on forward angle light scatter (FS) and side scatter (SS), and the proportions of cells that had lost FS and became small were determined in total populations of tumor cells. Untreated and DMSO treated tumors were used as controls. In addition, the proportions of cells that were stained with PI out of the total populations were also determined. Antibodies used for flow cytometry were purchased from BioLegend (San Diego, Calif.). Propidium iodide (PI) was purchased from Sigma-Aldrich (St. Louis, Mo.). WIN 55,212-2 mesylate was purchased from Tocris Bioscience (Bristol, UK).

### Example 2: WIN 55,212-2 Kills More OSCSCs than OSCCs

As shown in FIG. 1A-1E, OSCSCs are affected more by WIN 55,212-2 than OSCCs. Both OSCCs (oral squamous carcinoma cells) and OSCSCs (oral squamous carcinoma stem cells) were treated with various concentrations of WIN 55,212-2 (10, 25, 50, and 100 μM) and CDDP (50 μg/ml), and the cell numbers were determined after 24 hours of treatment.

OSCCs were killed significantly with CDDP but much less with different concentrations of WIN 55,212-2, whereas OSCSCs were not killed with CDDP but they were significantly killed with WIN 55,212-2. In OSCCs, healthy cells with more of the spindle shape were observed with treatment of WIN 55,212-2 of 50 μM or higher along with some rounded cells. In OCSCSs, the majority of the cells appeared abnormal in relation to the control cells. even at the lowest concentrations of WIN 55,212-2. CDDP had higher levels of toxicity to OSCCs than to OSCSCs. Percent decrease in cell numbers was calculated based on the control group for both OSCCs and OSCSCs after the cells were treated with different concentrations of WIN 55,212-2 and CDDP (50 μg/ml) for 24 hours (FIG. 1A) and 48 hours (FIG. 1B) and 24 hours (FIG. 1C) (n=3).

As shown in this figure, there is a smaller decrease in cell numbers of OSCCs when treated with various concentrations of WIN 55,212-2, whereas, there are greater decreases in cell numbers when treated with CDDP, which is known to be highly toxic to OSCCs. On the other hand, the percent decrease in cell numbers of OSCSCs are significantly greater than OSCCs when treated with WIN 55,212-2, and the levels were either lower or decrease to the levels caused by CDDP at the higher concentrations of WIN 55,212-2. Decrease in cell numbers was calculated based on the control group for both OSCCs and OSCSCs after the cells were treated with various concentrations of WIN 55,212-2 and CDDP (50 μg/ml).

Similar to the morphological images in FIG. 1A, there is much smaller decrease in the cell numbers when these cells were treated with different concentrations of WIN 55,212-2, but a significant decrease was seen when treated with CDDP, whereas OSCSCs treated with WIN 55,212-2 decreased the cell numbers substantially, but were changed less with CDDP. Therefore, there are significant differences in the response of OSCCs and OSCSCs to cannabinoids.

Overall these studies show that cannabinoids affect cancer stem cells more than differentiated tumors whereas CDDP, a chemotherapeutic drug, affects differentiated tumors more than cancer stem cells. Thus, cannabinoids and CDDP have cytostatic and cytotoxic effects on both differentiated and cancer stem cells, albeit with different degrees.

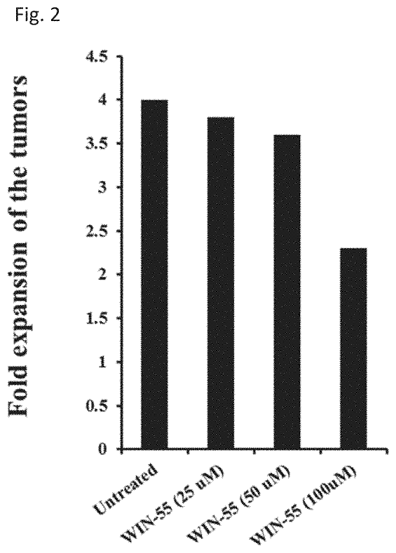

In FIG. 2, the cystostatic effect of WIN 55,212-2 are shown. WIN 55,212-2, at higher concentrations, arrests the tumor growth. The Y-axis shows the fold change of the tumor-cell coverage of the well-plate.

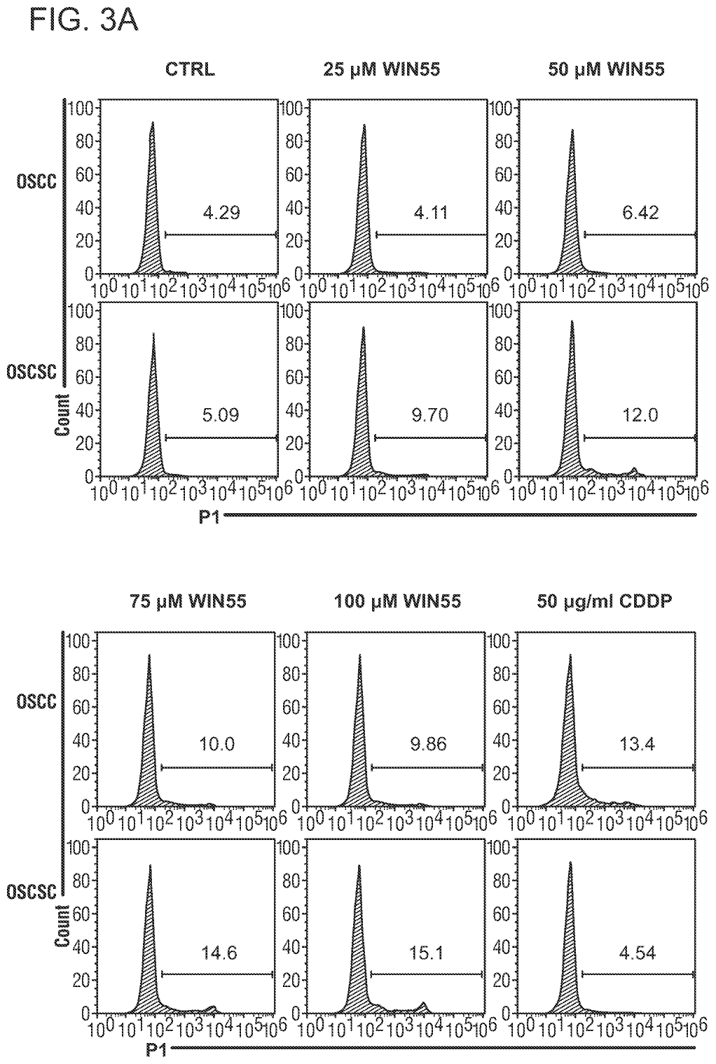

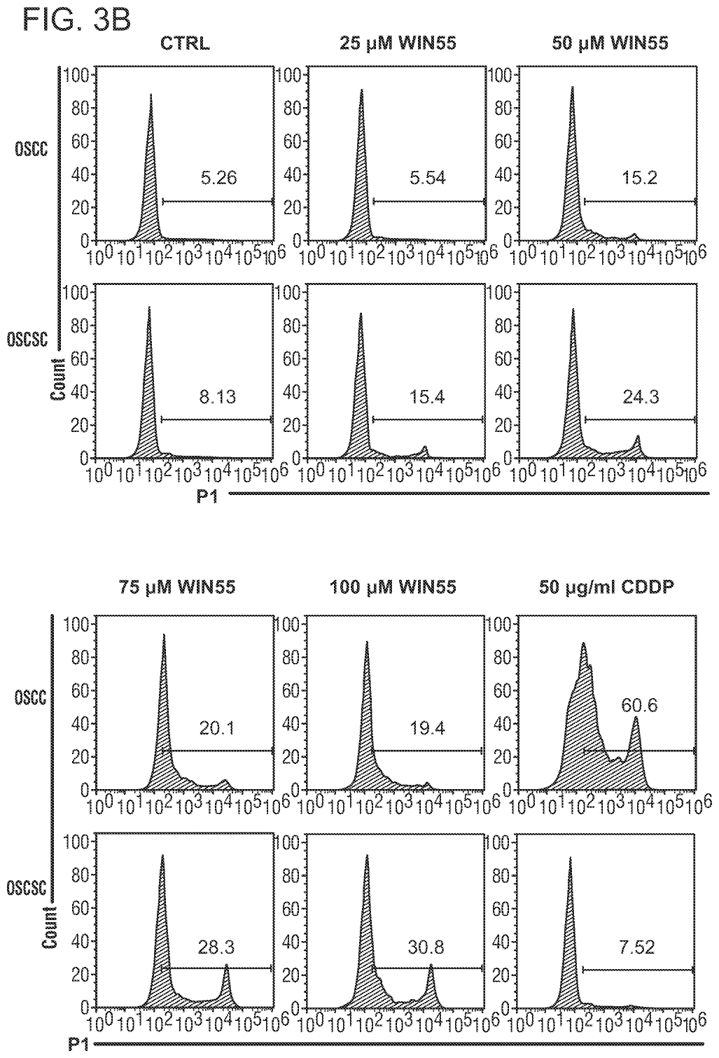

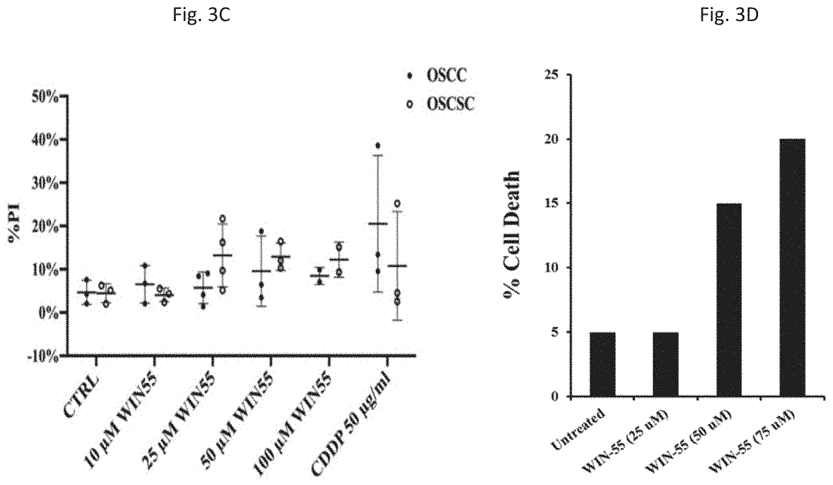

FIG. 3A-3D show that OSCSCs are affected by WIN 55,212-2, but not CDDP. OSCSCs and OSCCs were treated with WIN 55,212-2 at 25, 50, 75 and 100 μM and CDDP at 50 μg/ml for 24 hours.

OSCCs and OSCSCs were treated with various concentrations of WIN 55,212-2 for 24 hours (FIG. 3A) and 48 hours (FIG. 3B) and 24 hours (FIG. 3C) (n=2-4). Propidium iodide (PI) staining was used to determine the cell death by flow cytometry. Higher cell death was observed in OSCSCs when compared to OSCCs. Therefore, WIN 55,212-2 is a potent inducer of cell death in poorly differentiated oral tumors. In contrast, CDDP induced significant cell death in OSCCs when compared to OSCSCs. (FIG. 3D) is a regraphing of (FIG. 3C) but only for OSCSCs.

### Example 3: Decrease in Surface Proteins on OSCSCs and OSCCs Due to WIN 55,212-2 Treatment

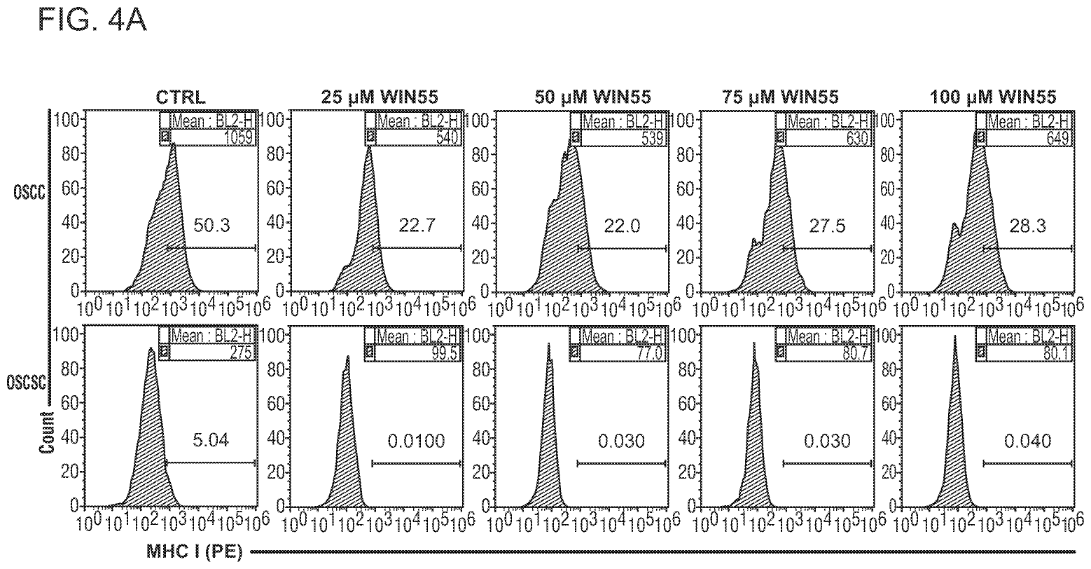

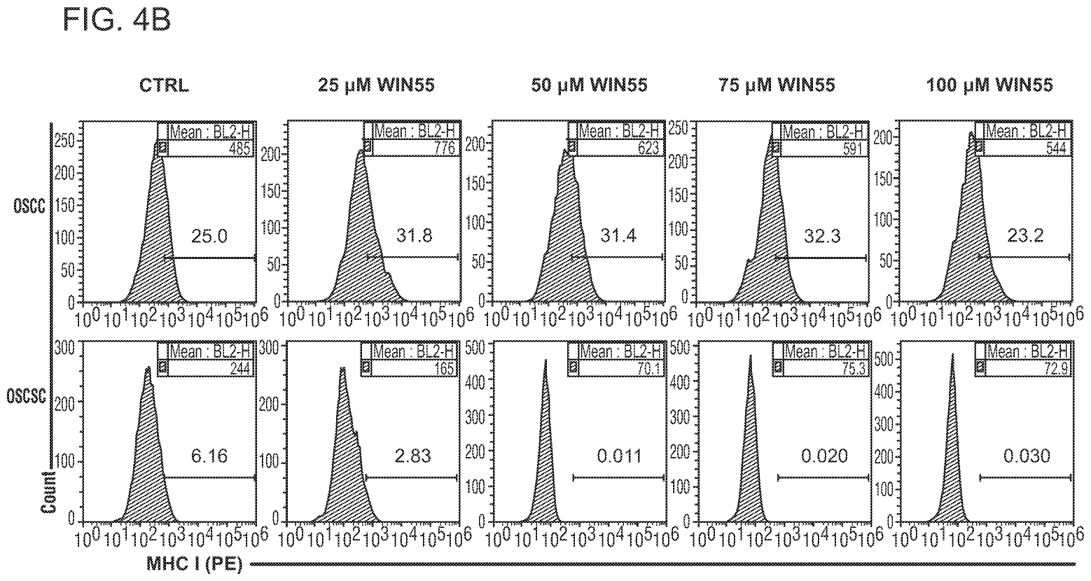

FIG. 4A-4B show that both OSCCS and OSCSCs exhibit a decrease in MHC-1 expression when treated with WIN 55,212-2. OSCCs and OSCSCs were treated with various concentrations of WIN 55,212-2 for 24 hours (FIG. 4A) and 48 hours (FIG. 4B) and the levels of MHC class I expression were determined on the cell surface after antibody staining followed by flow cytometric analysis. Both OSCCs and OSCSCs exhibited decreases in MHC class I expression after treatment with WIN 55,212-2. The decrease in MHC-1 will also allow for NK cytotoxicity.

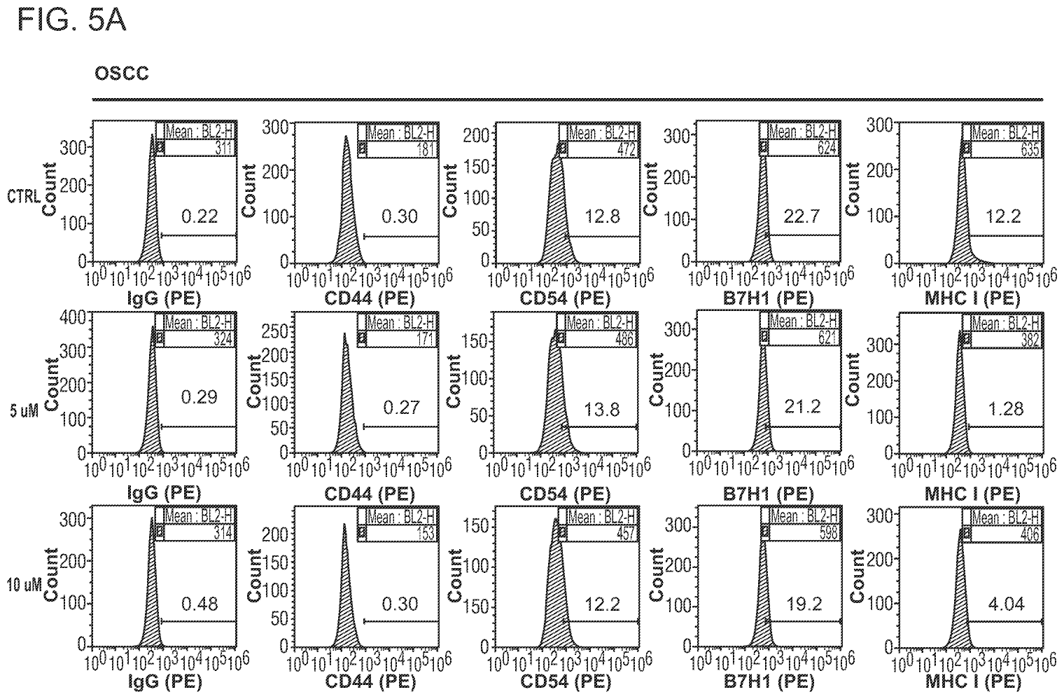

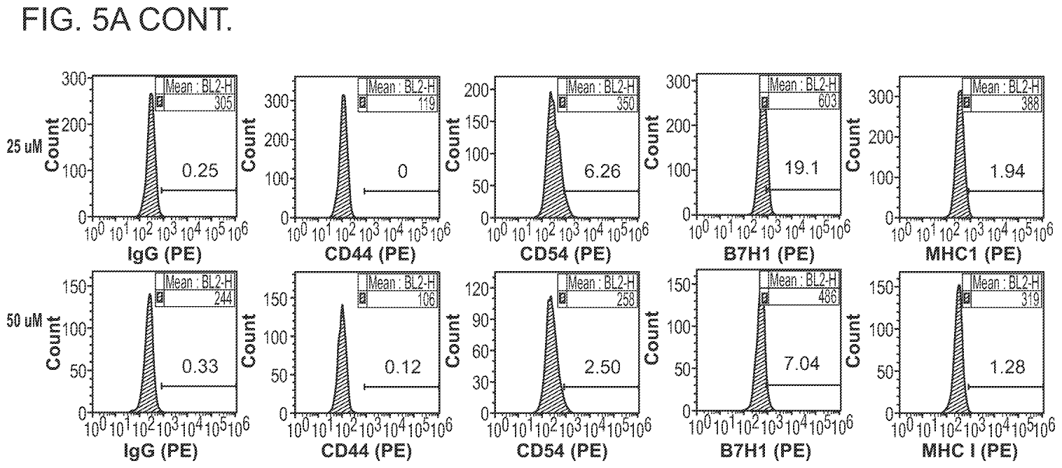

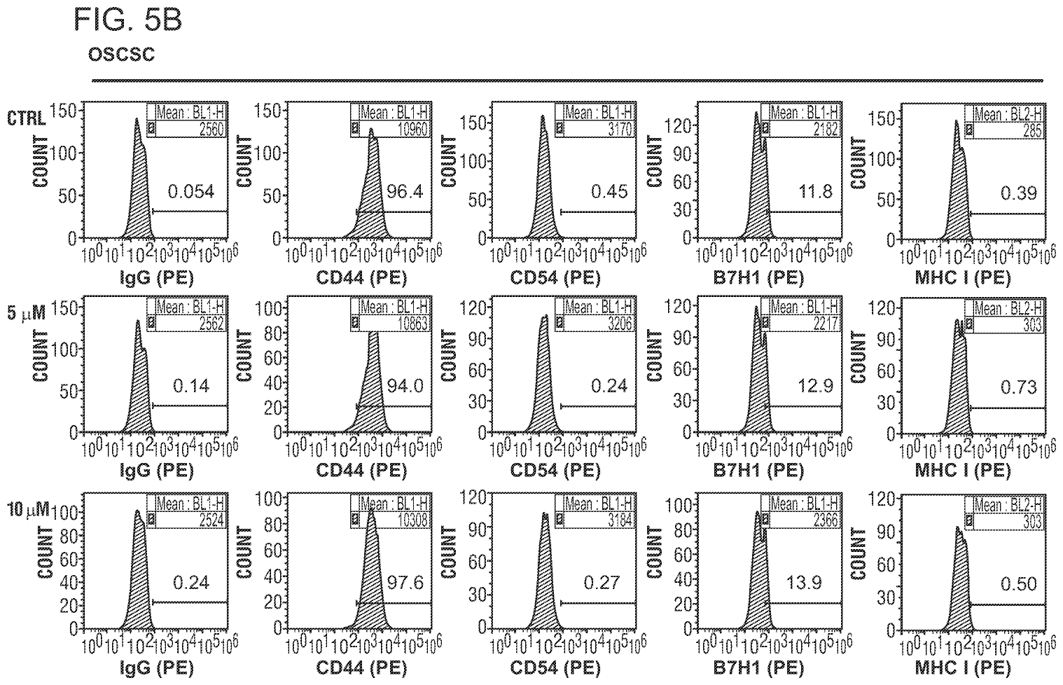

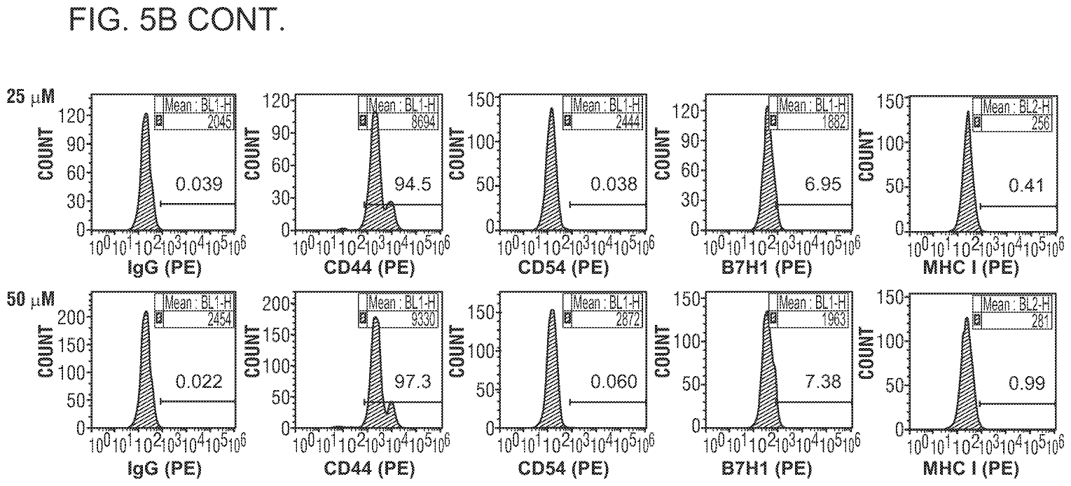

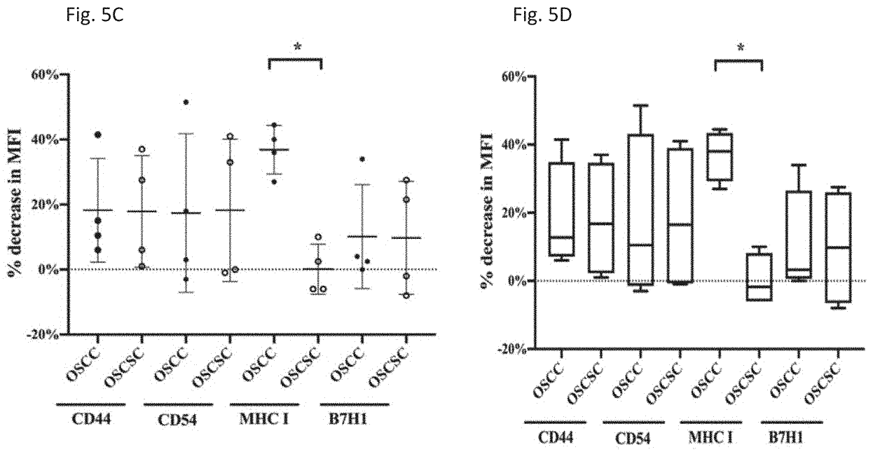

FIG. 5A-5D show that OSCCs and OSCSCs exhibit a decrease in the levels of surface receptor expression for CD44, CD54, B7H1 and MHC class I after 24-hour treatment of WIN 55,212-2. OSCCs (FIG. 5A) and OSCSCs (FIG. 5B) were treated with WIN 55,212-2 for 24 hours and the levels of surface receptor expression for CD44, CD54, B7H1, and MHC class I were determined after antibody staining followed by flow cytometric analysis. Both OSCCs and OSCSCs had decreased expressions of CD44, CD54, B7H1 and MHC class I after treatment with WIN 55,212-2. The data is graphed in FIG. 5C and FIG. 5D.

The decrease in these surface receptors is correlated with increased cytotoxicity by NK cells.

### Example 4: WIN 55,212-2 Kills More MP2s than PL12s

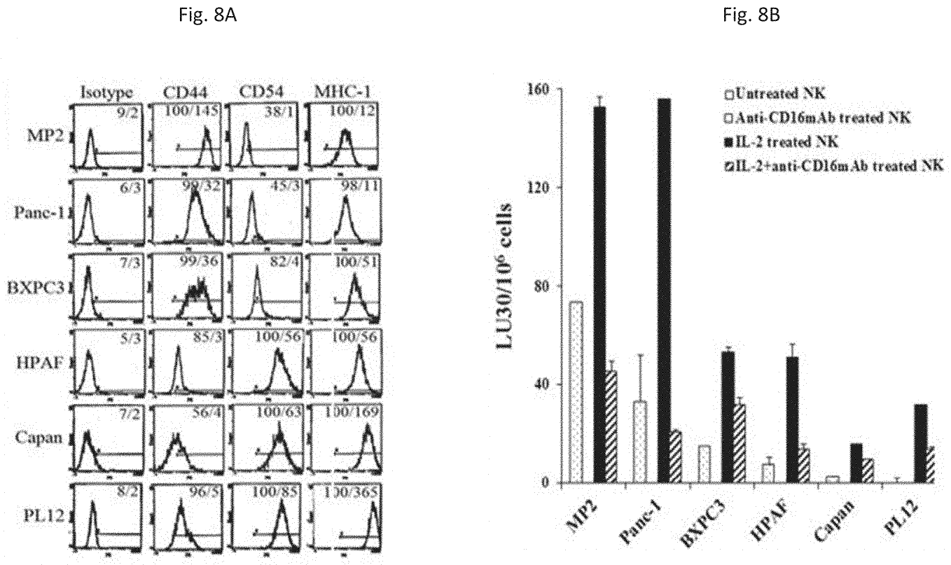

Six different pancreatic tumor cell lines each characterized at poorly, intermediate and well differentiated stages pathologically by other laboratories previously were used to determine phenotype, susceptibility to NK cell-mediated cytotoxicity and secretion of IFN-γ directly correlating with the differentiation stages of the tumors. Poorly-differentiated MP2 and Panc-1 demonstrated moderate to low levels of MHC-class I and CD54 in the presence of higher surface expression of CD44 receptors. Moderately differentiated BXPC3 and HPAF exhibited higher levels of MHC-class I surface expression in the presence of moderate to high expression of surface CD44 and CD54 receptors, and well-differentiated Capan and PL12 expressed higher levels of surface CD54 and MHC-class I in the presence of lower CD44 surface expression (FIG. 8A). Furthermore, the stage of differentiation of the tumors was correlated with sensitivity to NK cell mediated cytotoxicity in pancreatic tumor cells. The highest susceptibility to NK cell mediated cytotoxicity was seen with undifferentiated MP2 and Panc-1 tumors; whereas the well differentiated PL12 and Capan tumors demonstrated the lowest sensitivity to NK mediated lysis (FIG. 8B). BXPC3 and HPAF, being moderately differentiated tumors, exhibited intermediate sensitivity to NK cell lysis (FIG. 8B). Therefore, a direct correlation between augmented sensitivity to NK-mediated lysis and poor differentiation of pancreatic tumors was evident from these experiments. For the following experiments MP2 and PL12 were used as representative tumor types for poorly differentiated and well differentiated tumors respectively. Human pancreatic cancer cell line MIA PaCa-2 (MP2), was provided by Dr. Guido Eibl (UCLA David Geffen School of Medicine) and PL12 was provided by Dr. Nicholas Cacalano (UCLA Jonsson Comprehensive Cancer Center). MP2 tumors were cultured with DMEM in supplement with 10% FBS and 1% Penicillin-Streptomycin (Gemini Bio-Products, CA). PL12 pancreatic tumors were cultured in RMPI 1640 medium supplemented with 10% FBS and 1% penicillin-streptomycin.

FIG. 8A-8B show the stage of differentiation in pancreatic tumors correlated with susceptibility to NK cell-mediated cytotoxicity. The surface expression of CD44, CD54, and MHC-class I on pancreatic tumors were assessed by flow cytometry after staining with PE-conjugated antibodies. Isotype control antibodies were used to determine non-specific binding. Numbers in each histogram represent percent/Mean Channel Fluorescence (MFI) (FIG. 8A) Freshly isolated NK cells were left untreated or treated with anti-CD16 mAb (3 μg/mL), IL-2 (1000 U/mL) or the combination of anti-CD16 mAb (3 μg/mL) and IL-2 (1000 U/mL) for 18 h before they were used in co-cultures with ⁵¹Cr labeled MP2, Panc-1, BXPC3, HPAF, Capan and PL12. NK cell-mediated cytotoxicity was determined using 4-hour ⁵¹Cr release assay, and the lytic units 30/106 cells were determined using inverse number of NK cells required to lyse 30% of the target cells×100. (FIG. 8B) One of several representative experiments is shown in the figure.

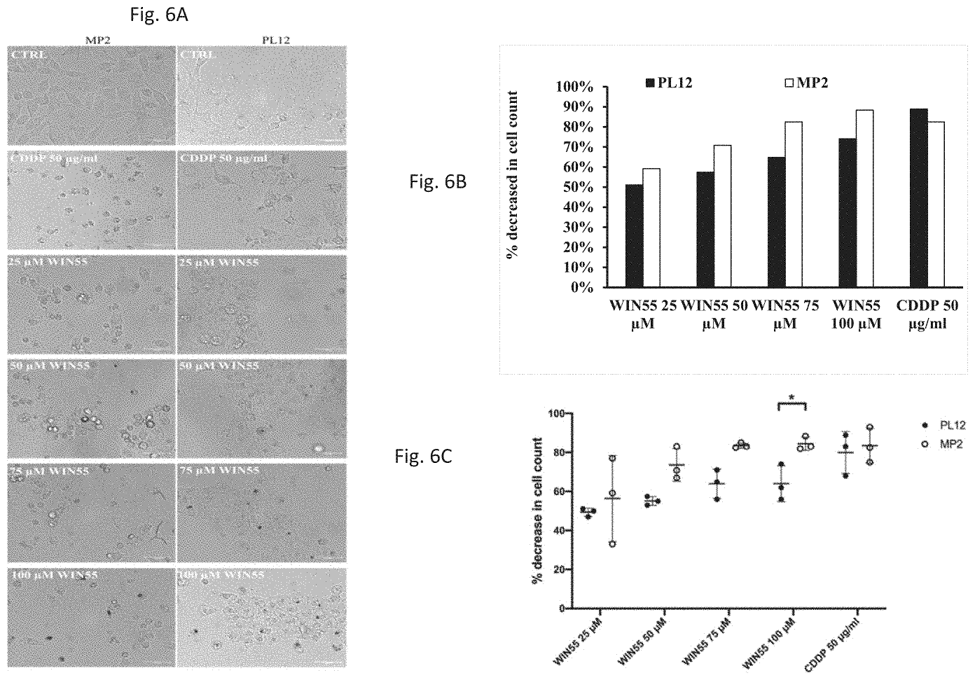

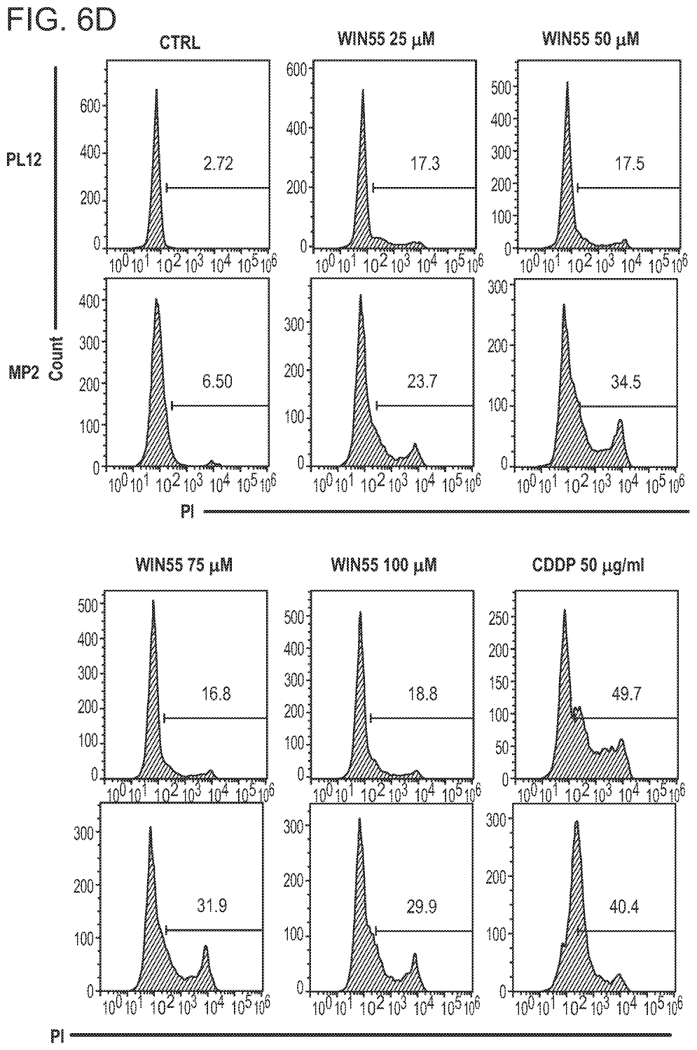

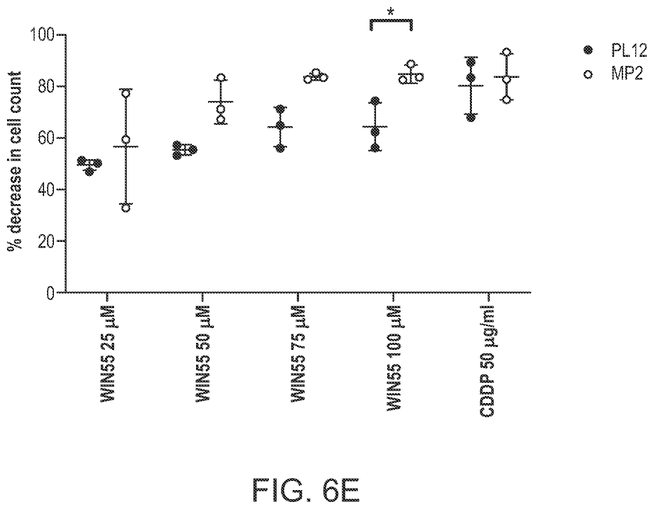

FIG. 6A-6E show that PL12 tumors had a lower decrease in cell count when treated with WIN 55,212-2 when compared to MP2 tumors. MP2 and PL12 pancreatic tumors were treated with WIN 55,212-2 for 24 hours before the pictures were taken. In PL12, more tumors were alive when treated with WIN 55,212-2 when compared to MP2 tumors.

In FIG. 6B and FIG. 6C, the percent decrease in cell numbers was calculated based on the control group for both MP2 and PL12 after the cells were treated with various concentrations of WIN 55,212-2 and CDDP (50 μg/ml). A dose dependent decrease in cell numbers in both PL-12 and MP2 can be seen after the treatment of the cells with WIN 55,212-2. There are smaller decreases in cell numbers of PL12 when treated with various concentrations of WIN 55,212-2, whereas, there is a greater decrease in cell numbers when treated with CDDP, which is known to be toxic to PL12. In contrast, the percent decrease in cell numbers of MP2s are significantly greater than PL12s when treated with WIN 55,212-2, and the levels reach to the levels of killing seen by CDDP at the higher concentrations of WIN 55,212-2.

FIG. 6D and FIG. 6E show that MP2s and PL12s treated with various concentrations of WIN 55,212-2 for 48 hours. Propidium iodide (PI) staining was used to determine the cell death by flow cytometry. A greater higher increase in cell death was observed in MP2s when compared to PL12, in a dose dependent manner. Therefore, WIN 55,212-2 is a potent inducer of cell death in poorly differentiated MP2 tumors.

### Example 5: Decrease in Surface Proteins on MP2 and PL12 Due to WIN 55,212-2 Treatment

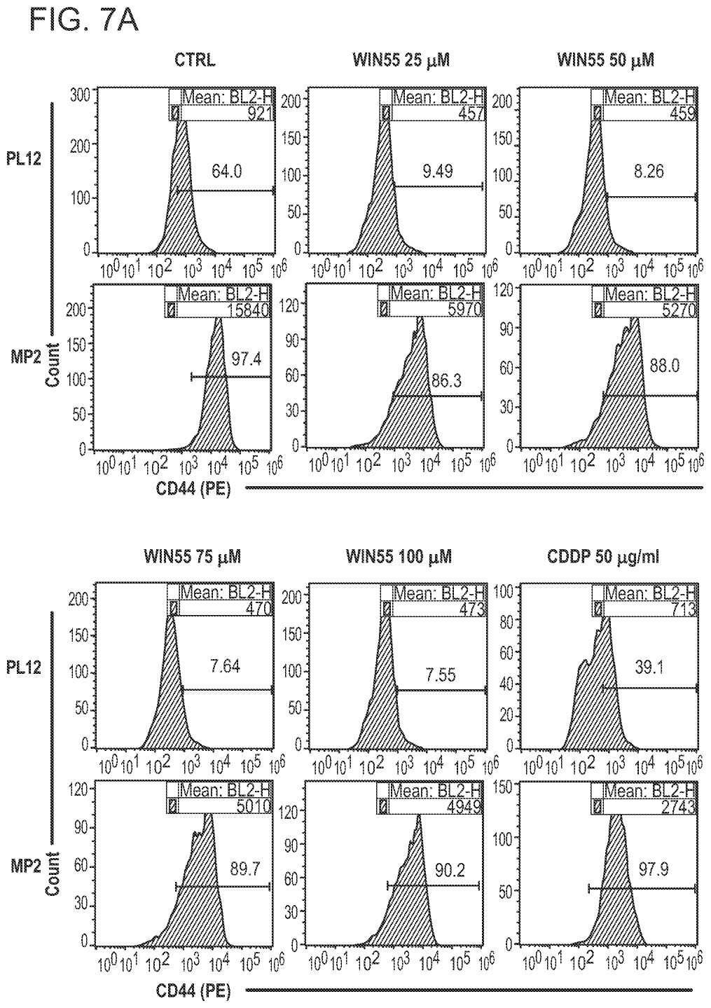

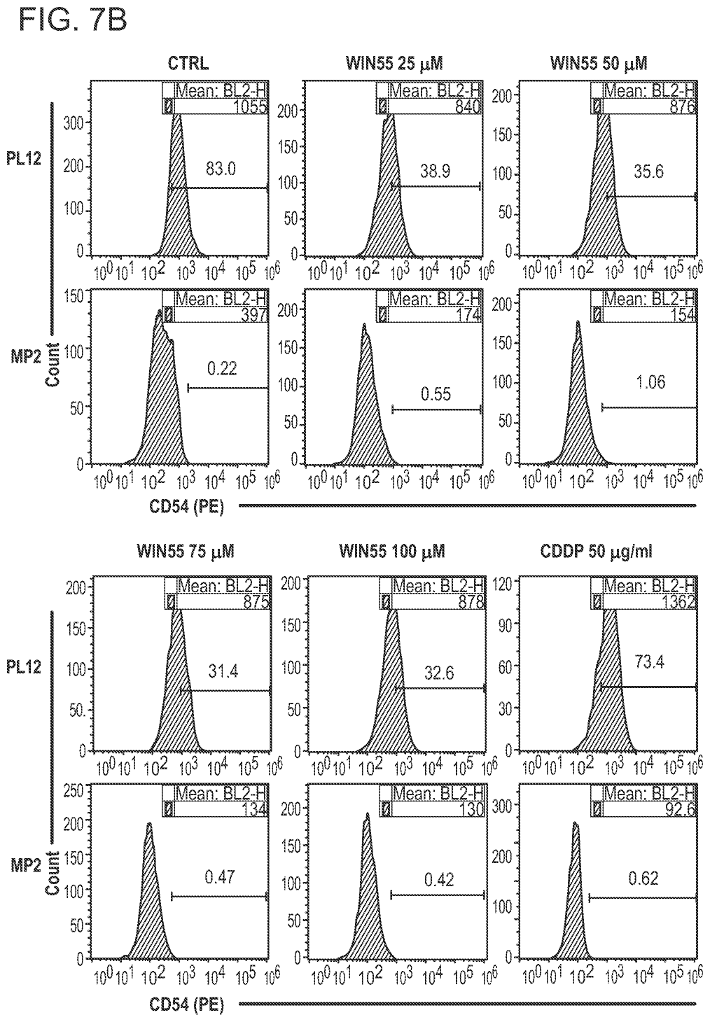

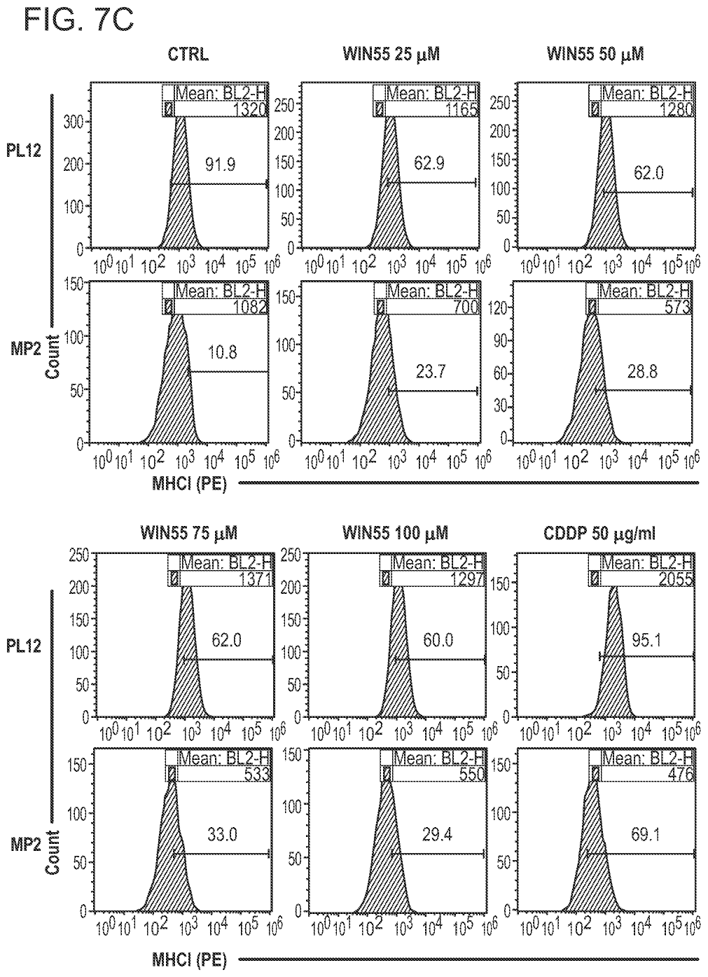

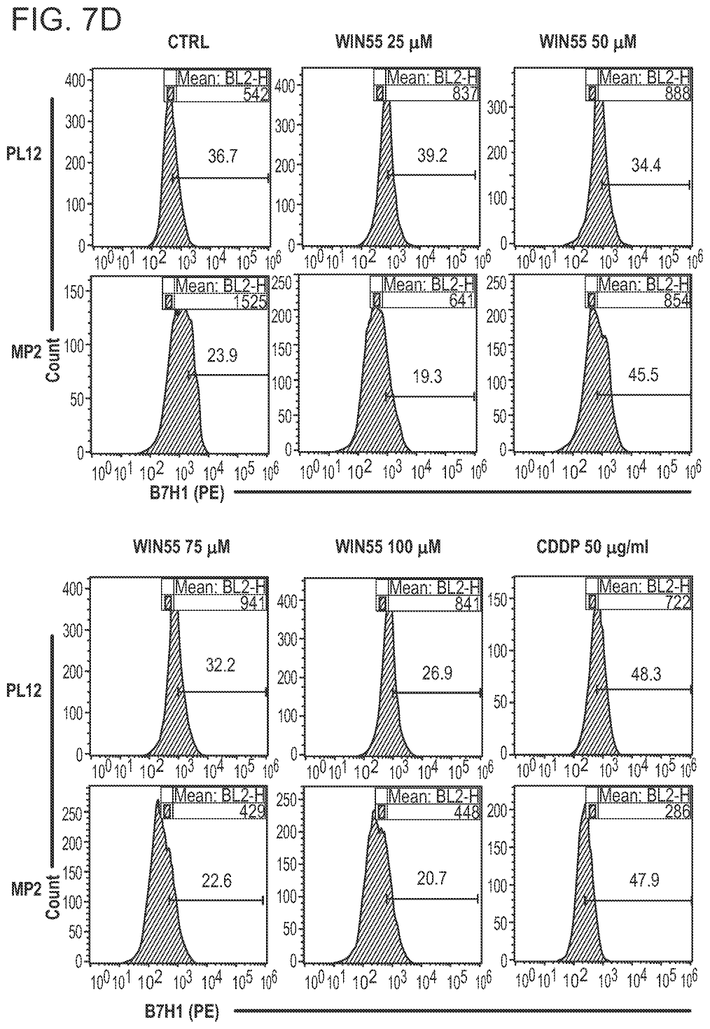

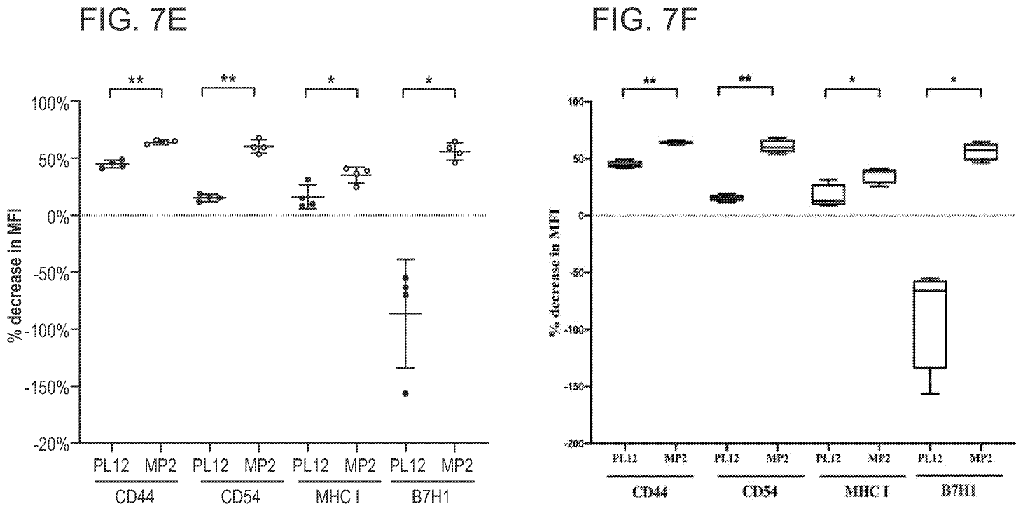

FIG. 7A-7F show that PL12 and MP2 pancreatic tumors exhibited lower levels of CD44, CD54, MHC class I, and B7H1 surface antigens after treatment with WIN 55,212-2 for 48 hours. The levels of surface receptor expression for CD44 (FIG. 7A), CD54 (FIG. 7B), MHC class I (FIG. 7C) and B7H1 (FIG. 7D) were determined after antibody staining followed by flow cytometric analysis. Both PL12 and MP2 tumors had decreased expressions of CD44, CD54, B7H1 and MHC class I after treatment with WIN 55,212-2, albeit the decrease for MP2s were greater than PL12s. The data is graphed in FIGS. 7E and 7F.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

### Example 6: Materials and Methods for Example 7

### Cell Lines and Reagents

RPMI 1640 (Gibco, Thermo Fisher Scientific, USA) complete medium with 10% fetal bovine serum (FBS) (Gemini Bio-Products, San Diego, Calif., USA), 1.4% of non-essential amino acid (Gibco, Thermo Fisher Scientific, USA), 1.4% sodium pyruvate (Gibco, Thermo Fisher Scientific, USA), 0,15% of sodium bicarbonate (Fisher Scientific, Waltham, Mass., USA) and 1% antibiotics/antimycotics (Gemini Bio-Products, San Diego, Calif., USA) was used for oral tumor culture. DMEM (Gibco, Thermo Fisher Scientific, USA) supplemented with 10% FBS and 1% antibiotics/antimycotics was used for pancreatic tumor cell culture. Oral squamous carcinoma cells (OSCCs) and oral squamous carcinoma stem cells (OSCSCs) were isolated from cancer patients with tongue tumor at UCLA. Human pancreatic cancer cell lines MIA PaCa-2 (MP2) and PL-12 were generously provided by Dr. Guido Eibl (UCLA David Geffen School of Medicine) and by Dr. Nicholas Cacalano (UCLA Jonsson Comprehensive Cancer Center), respectively. Antibodies to CD44, MHC Class-I, CD54 and PD-L1 used for flow cytometry were purchased from BioLegend (San Diego, Calif.). Propidium iodide (PI) was purchased from Sigma-Aldrich (St. Louis, Mo.). WIN 55,212-2 mesylate was purchased from Tocris Bioscience (Bristol, UK). CDDP was purchased from Ronald Reagan UCLA Medical Center Pharmacy. PE conjugated CB1R and Alexa fluor 488 conjugated CB2R antibodies were purchased from Biotechne (NE, MN). TNF-α and IFN-γ were purchased from Peprotech (Rockyhill, N.J.).

### Microscopy

Tumor cells were cultured at 3×10⁵ cells per ml in 12 well plates and treated with different concentrations of WIN 55,212-2 and CDDP as described in the figure legends and images of the cells were taken under 400× magnification using DMI6000 B inverted microscope and LAS X software (both Leica, Wetzlar, Germany).

### Cell Count

Tumor cells were cultured at 3×10⁵ cells per ml in 12 well plates and treated with different concentrations of WIN 55,212-2 and CDDP as described in the figure legends, and the detached cells were collected before the wells were washed with 1×PBS and the attached cells were harvested with trypsin-0.25% EDTA (Gibco, Thermo Fisher Scientific, PA, USA) and they were combined with detached cells and counted. The number of viable cells was counted under light microscope using Trypan Blue staining (Sigma, Mo., USA).

### Surface Staining and Cell Death Assays

Tumor cells were cultured at 3×10⁵ cells per ml in 12 well plates and treated with different concentrations of WIN 55,212-2 and CDDP as described in the figure legends, and the detached cells were collected before the wells were washed with 1×PBS and the attached cells were harvested with trypsin-0.25% EDTA (Gibco, Thermo Fisher Scientific, PA, USA) and they were combined with detached cells before they were stained with the antibodies and Propidium Iodide (PI) (Sigma, Aldrich). Cell surface receptor staining was performed by labeling the cells with PE-conjugated antibodies against CD44, CD54, PD-L1, CB1R and MHC class I or propidium iodide (PI) and Alexa fluor 488 conjugated CB2R as described previously. Attune NxT flow cytometer (Thermo Fisher Scientific, MA, USA) were used to run the samples and the results were analyzed using FlowJo vX software (BD, OR, USA).

### Statistical Analysis

A paired or unpaired, two-tailed Student t test was performed for the statistical analysis using Prism-7 software (Graphpad Prism, CA, USA) to compare different groups. The following symbols represent the levels of statistical significance within each analysis, *** (p-value<0.001), ** (p-value 0.001-0.01), * (p-value 0.01-0.05).

### ABBREVIATIONS

CSC cancer stem cells
MHC class I Major Histocompatibility Complex Class I
PD-L1 Program Death Ligand 1
CBD Cannabidiol
THC Tetrahydrocannabinol
PD-1 Program Death-1
EDTA Ethylenediamine tetraacetic acid
OSCC Oral Squamous Carcinoma Cells
OSCSC Oral Squamous Carcinoma Stem Cells
CDDP cis-diamminedichloroplatinum(II)
MP2 Mia-PaCa2
PI Propidium Iodide

### Example 7: Synthetic Cannabinoid WIN 55,212-2 has a Profound Inhibitory Effect on Oral and Pancreatic Stem-Like Tumor Cells

It has been previously demonstrated that several carcinoma stem cells (CSCs) and poorly differentiated tumor cells were found to be resistant to the effects of chemotherapy, despite being highly susceptible to NK cell mediated cytotoxicity. In contrast, well differentiated tumor cells were more susceptible to chemotherapy and resistant to NK cell mediated killing. Here, the effect of a synthetic cannabinoid WIN 55,212-2 on tumor cell types in different stages of differentiation was studied. It is presented herein that WIN 55,212-2 inhibited tumor cell proliferation and induced cell death in oral and pancreatic tumor cells, and the effect was more pronounced in stem-like OSCSCs and MP2 cells as compared to well differentiated OSCCs, and PL-12 tumor cells. Overall, it was demonstrated herein that WIN 55,212-2 has a significant targeting activity against cancer stem cells/poorly differentiated tumor cells and this effect is more pronounced on stem-like tumor cells/poorly differentiated tumor cells than on differentiated tumor cells.

Cannabinoid-based drugs have been used as palliative treatments along with conventional therapy for amelioration of side effects of radio- and chemotherapy to reduce nausea and stimulate appetite in cancer patients. Cannabinoids were shown to act through activating cannabinoid receptors, CB1 and CB2. Both of these receptors were shown to be increased on tumor cells of multiple origin, including prostate, glioblastoma, hepatocarcinoma, breast and non-small cell lung cancer. Components of endo-cannabinoid system has been shown to have anti-tumor effects by inhibiting the proliferation and inducing cell death through apoptosis. WIN 55,212-2, a potent cannabinoid receptor agonist, was shown to mediate anti-tumor effect through inducing caspase-independent apoptosis and inhibiting migration and invasion of tumors in several studies, such as glioblastoma, renal cell carcinoma, hepatocellular carcinoma, osteosarcoma, tumorigenic epidermal tumors, prostate tumors, human Kaposi's sarcoma tumors, mantle cell lymphoma, melanoma, and breast cancers. It was also reported that WIN 55,212-2 synergistically increased the effects of radiotherapy in breast cancer cell lines but not in normal breast epithelium, whereas other cannabinoids such as CBD, nabilone and THC failed to enhance anti-proliferative effects of radiation. Furthermore, WIN 55,212-2 was shown to reduce tumor burden, lung metastasis and tumor induced angiogenesis in vivo in mouse models of breast cancer, non-small cell lung cancer, and non-melanoma skin cancer.

Due to significant heterogeneity in tumor cells it is quite challenging to eliminate tumors and predict the disease outcome. Despite numerous studies on the anti-tumor effect of WIN 55,212-2, its effect on differentiated vs stem-like tumor cells still remains to be elucidated. Four surface receptor antigens, CD44, CD54, PD-L1 and MHC class I, mark the differentiation status of tumor cells. Specifically, CD44 was shown to be increased on the surface of stem-like tumors and decreased on the surface of differentiated tumors, whereas, CD54, PD-L1, and MHC class I were found to be elevated on the surface of differentiated tumors. In this study, these four surface markers were used to investigate the effects of WIN 55,212-2 on poorly differentiated/cancer stem like cells (CSCs) and their counterpart, differentiated tumor cells in two tumor types, oral and pancreatic tumors. It is also demonstrated herein that the anti-proliferative and cytotoxic effect of WIN 55,212-2 are more pronounced on poorly differentiated/CSC tumors when compared to well-differentiated counterparts, even though they can also inhibit the growth and proliferation of well-differentiated tumors.

### WIN 55,212-2 Inhibited Cell Proliferation and Induced Cell Death in Oral Tumor Cells, and the Effect was More Pronounced in Stem-Like OSCSCs

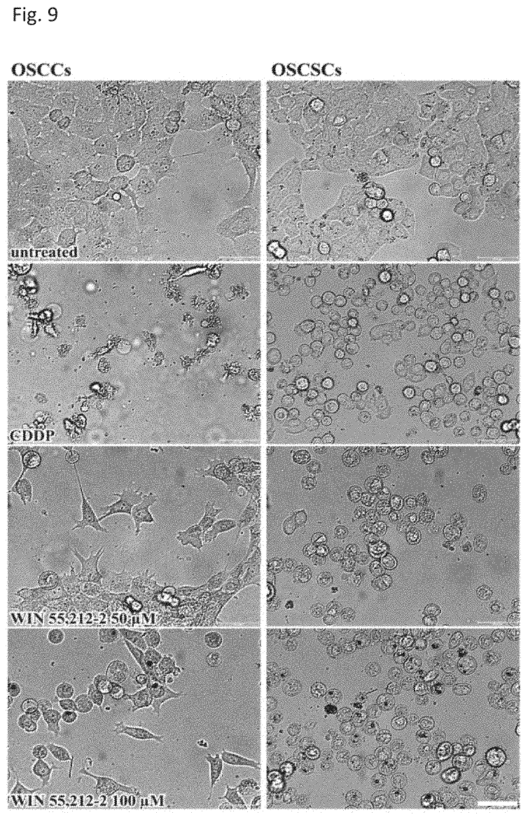

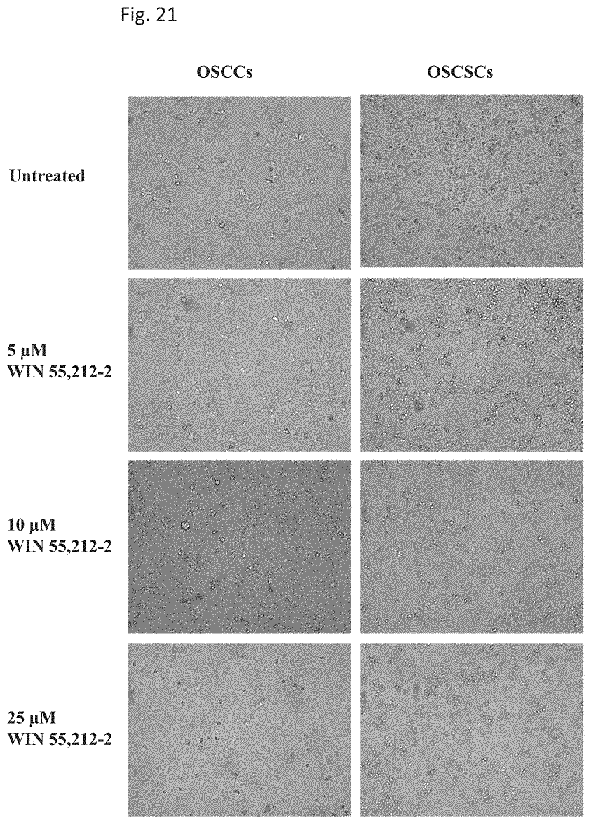

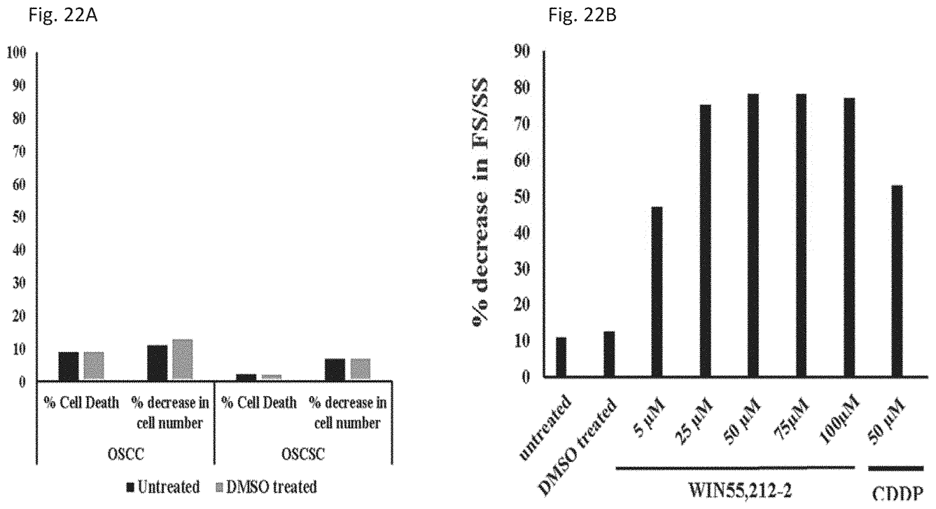

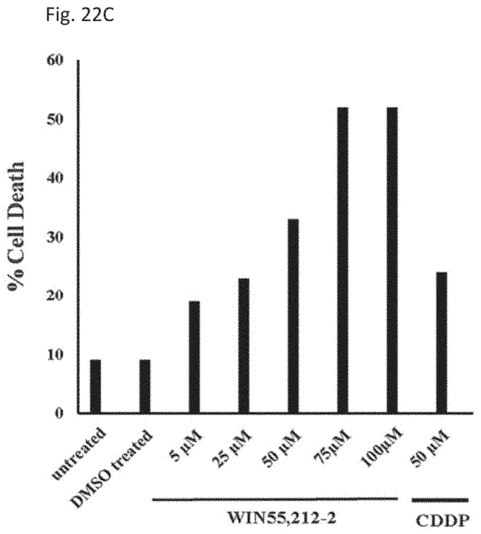

Oral squamous carcinoma cells (OSCCs) and oral squamous carcinoma stem like tumor cells (OSCSCs) were treated with different concentrations of WIN 55,212-2 (50-100 μM) and cis-diamminedichloridoplatinum(II) (CDDP) (50 μg/mL) for 24 h. CDDP, a chemotherapeutic drug has shown to be more cytotoxic to well-differentiated tumor cells, was used as a positive control for cell death evaluation here. When treated with low concentrations of WIN 55,212-2, OSCCs remained viable (FIG. 21). After treatment with 50 μM or higher concentrations of WIN 55,212-2, some of the OSCCs still remained viable but some rounded up and detached and were not viable. OSCSCs, the stem-like tumor cells, became detached from the culture plates when treated with WIN 55,212-2, even at the lowest concentrations. On the other hand, CDDP caused OSCCs' loss of morphology, and the majority were detached from the plates. Despite the loss of some morphology in OSCSCs, the majority of these cells were still attached to the cell culture plate after CDDP treatment (FIG. 9).

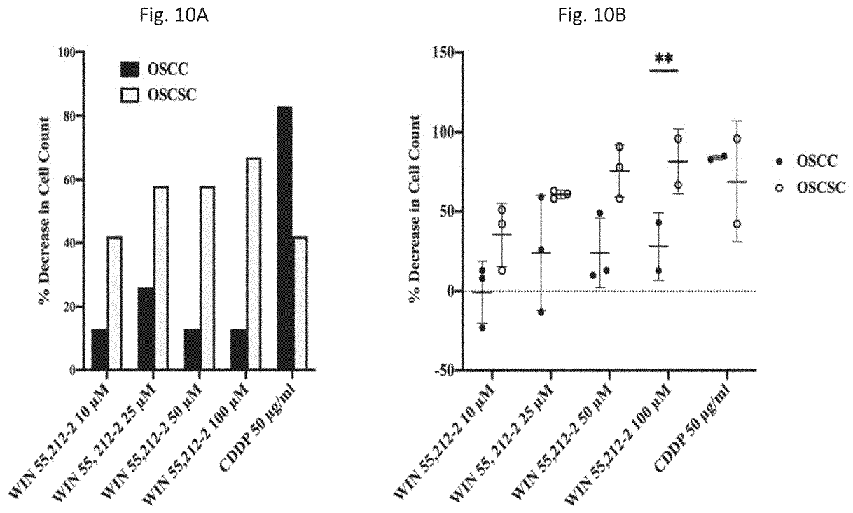

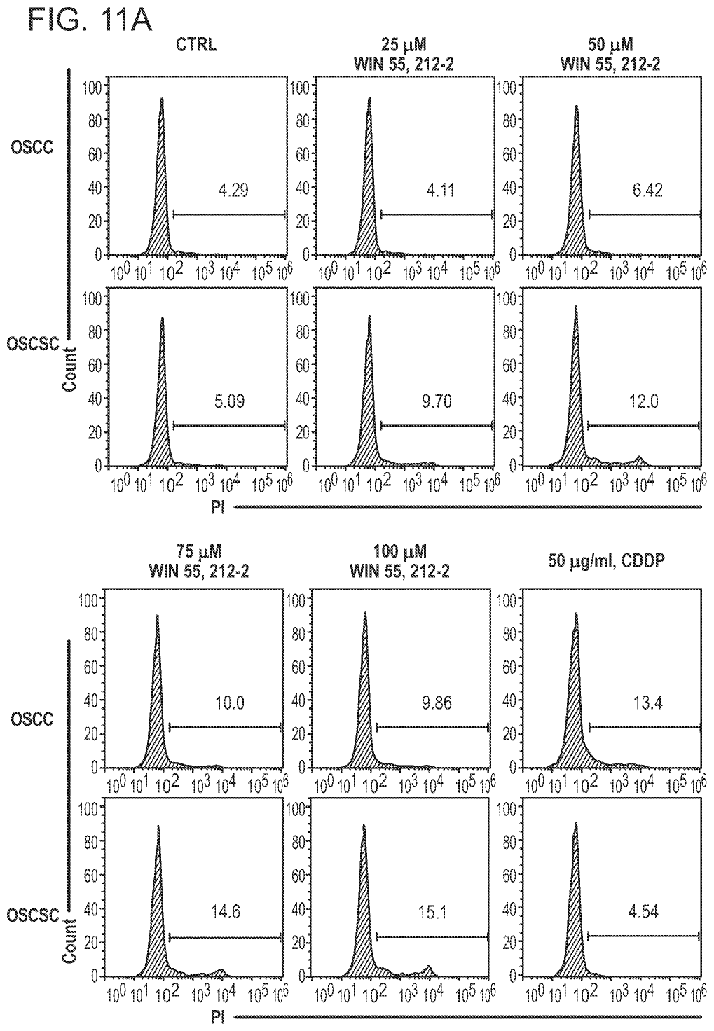

Cell growth of OSCCs and OSCSCs was evaluated after WIN 55,212-2 (10-100 μM) and CDDP (50 μg/mL) treatments by counting the numbers of viable cells using microscopy. WIN 55,212-2 decreased the cell numbers of OSCC by 10-25% after 24 hours of treatment, whereas a greater decrease was observed in the cell numbers of OSCSC, ranging from 40% to 65%. In contrast, CDDP was found to inhibit cell growth of OSCCs more than those seen with OSCSCs (FIGS. 10A, 10B).

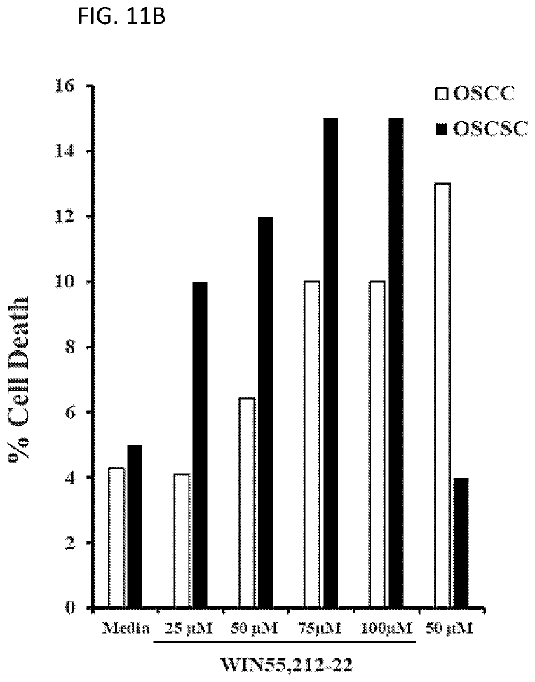

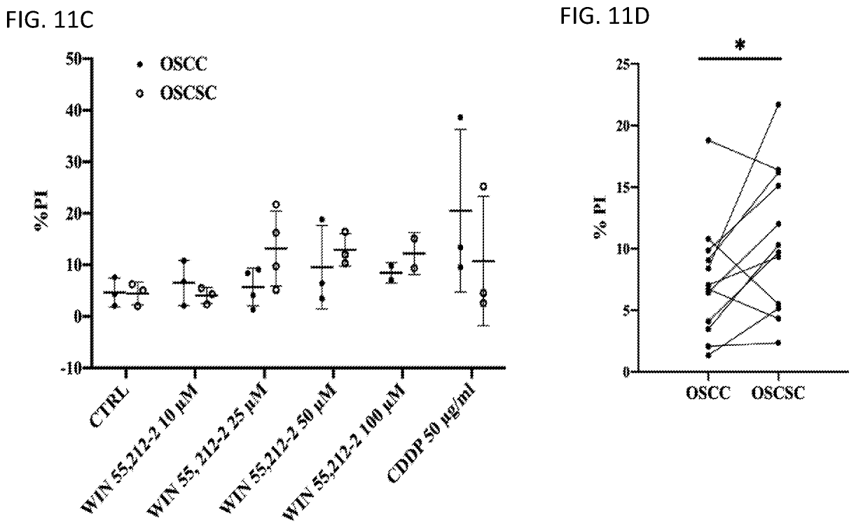

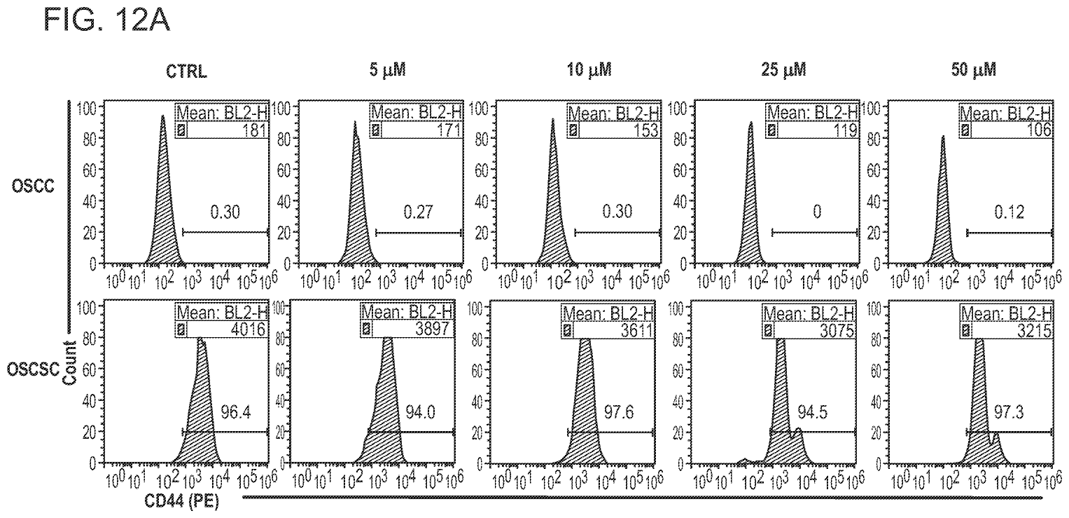

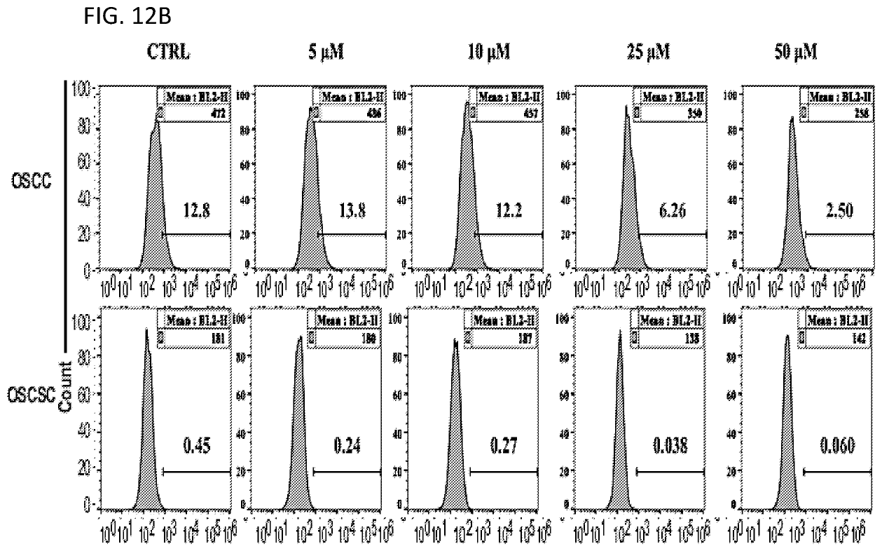

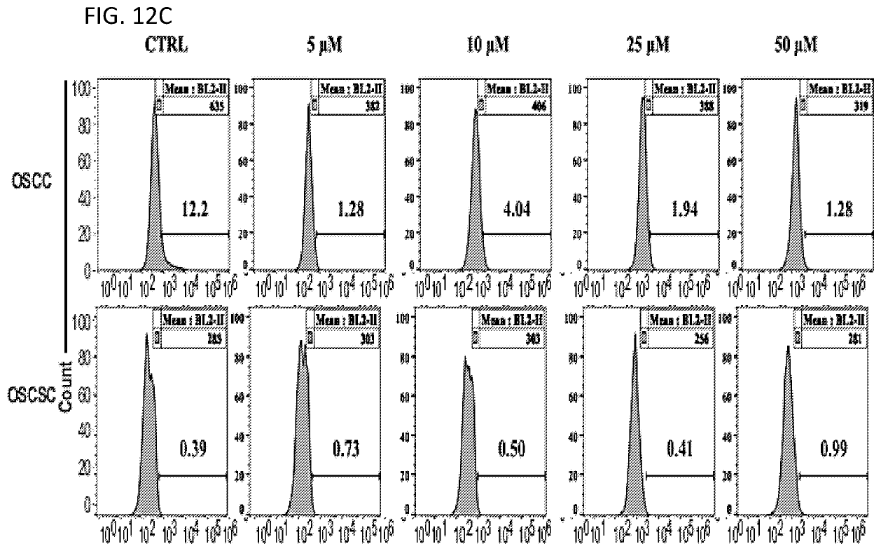

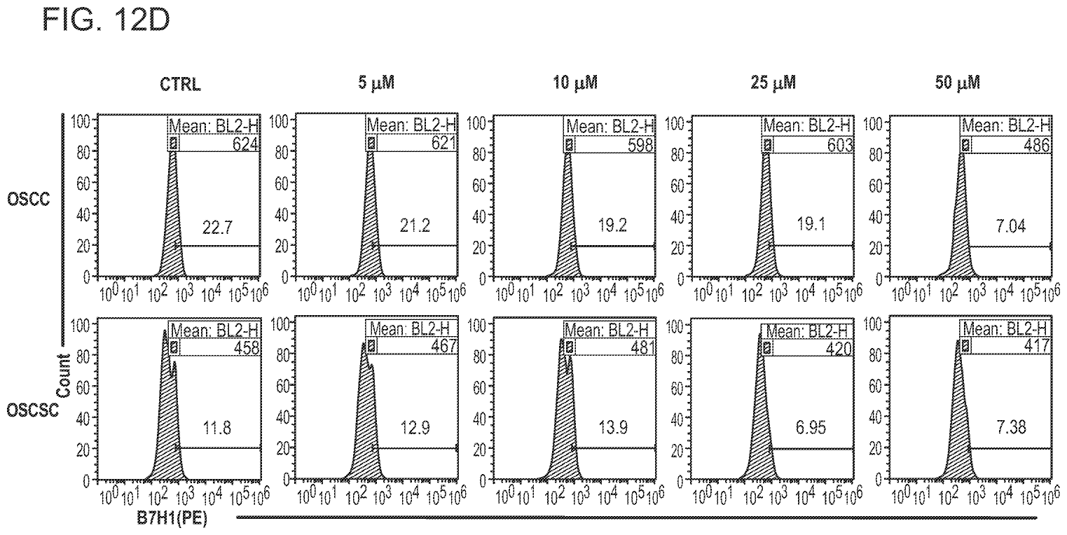

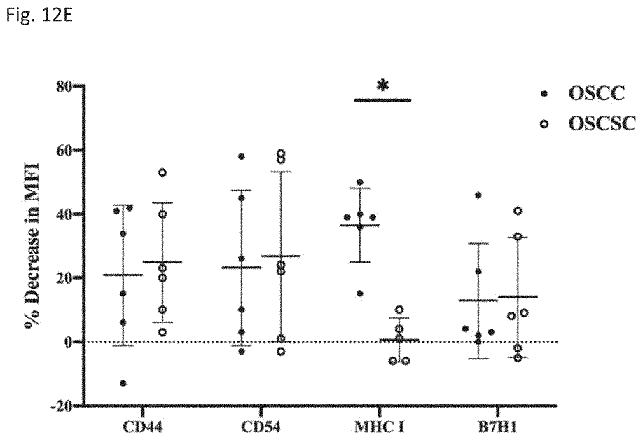

Cell death was then evaluated after WIN 55,212-2 and CDDP treatment by using PI staining and flow cytometric analysis. Concentrations of 75 and 100 μM WIN 55,212-2 induced cell death in OSCCs up to 10% after 24 hours of treatment, however, higher cell death was detected in OSCSCs at all concentrations tested (FIG. 11B). As expected, CDDP triggered more cell death in OSCCs when compared to OSCSCs (FIG. 11B). Although on average, higher cell death was observed in OSCSCs by WIN 55,212-2 treatment when compared to OSCCs, when considering the cumulative effect of all the experiments, the values obtained did not achieve statistical significance. The average amount of cell death induced by CDDP was higher in OSCCs when compared to OSCSCs, and the amounts of cell death induced at higher concentrations of WIN 55,212-2 (25-100 μM) in OSCSCs were either similar or higher when compared to those induced by CDDP (FIGS. 11C, 11D). Therefore, WIN 55,212-2 is a potent inducer of cell death in poorly differentiated oral tumor cells.

### WIN 55,212-2 Decreased Cell Surface Expression of CD44, CD54, MHC Class I and PD-L1 in Oral Tumor Cells

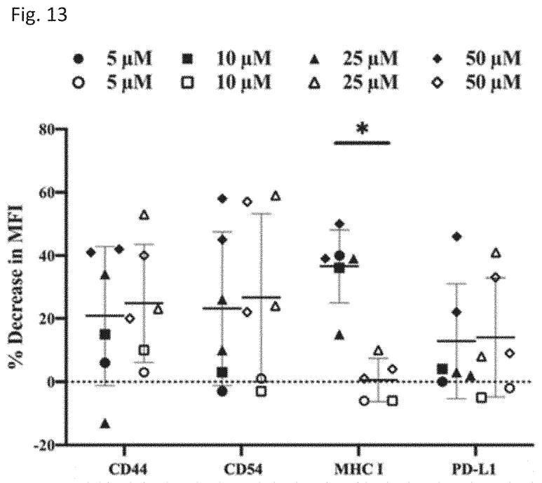

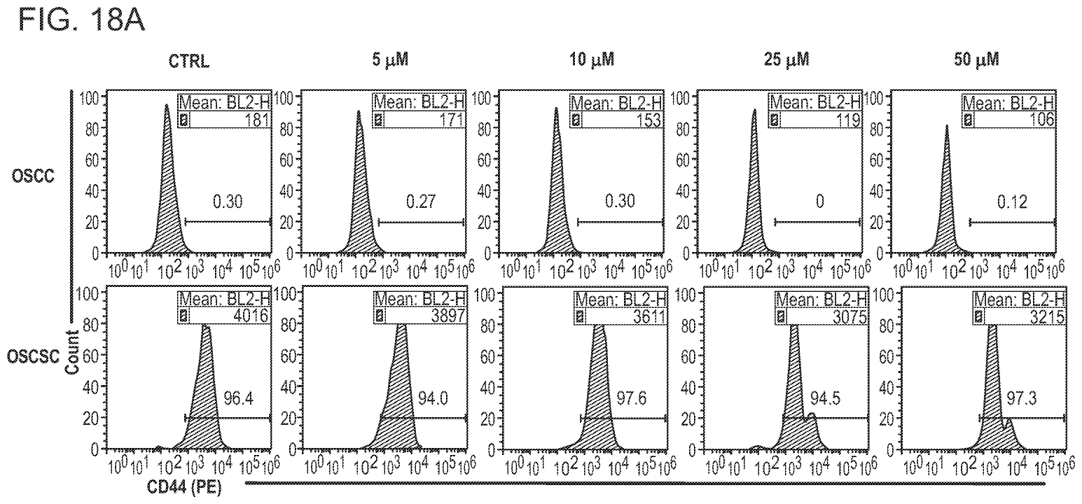

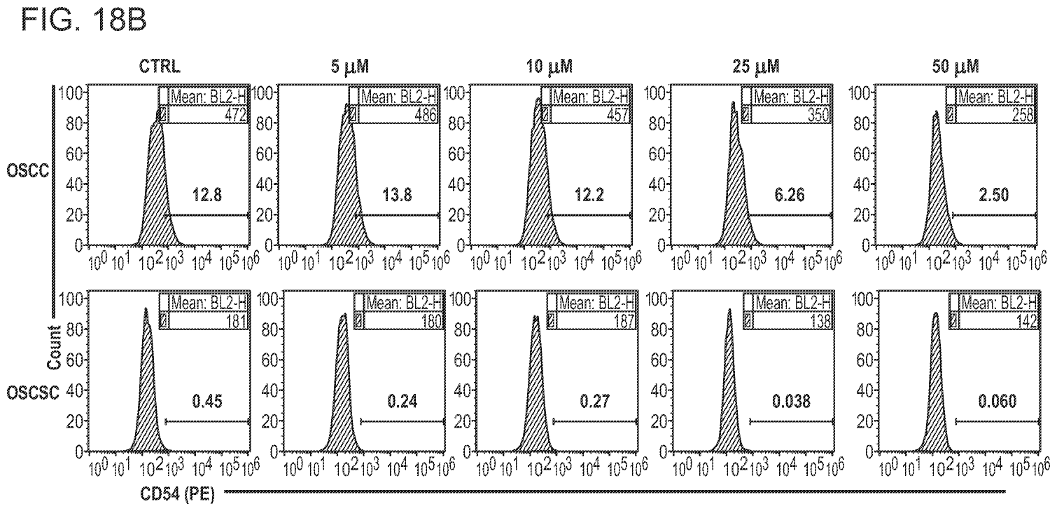

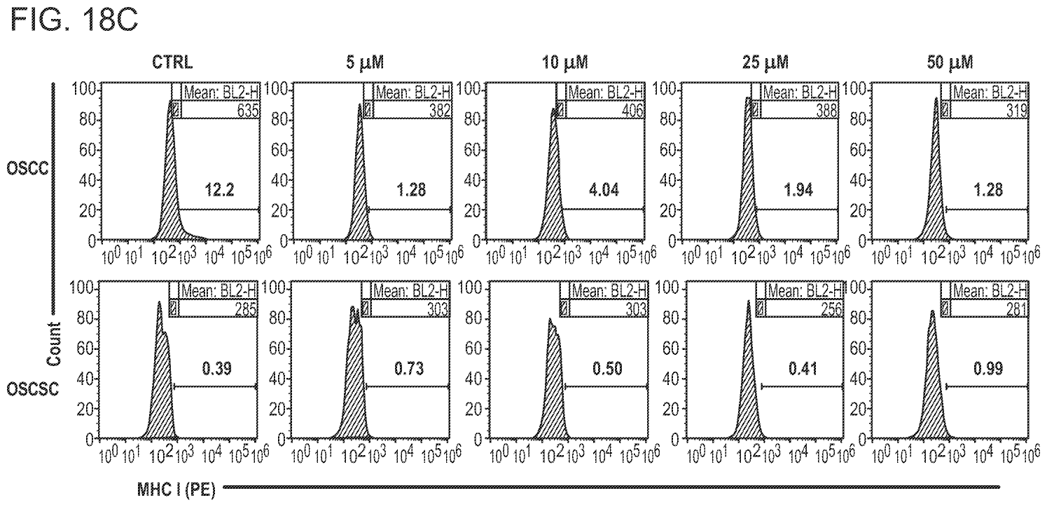

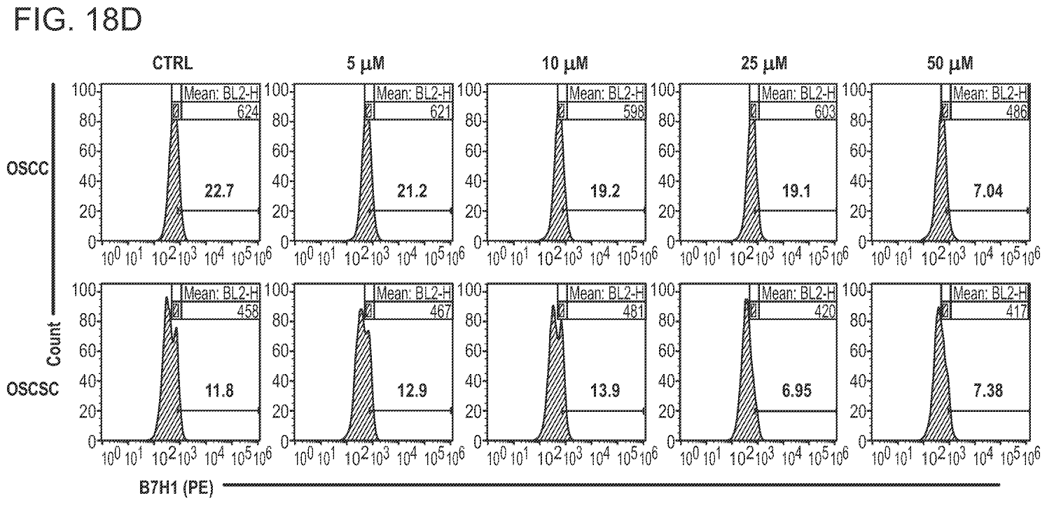

Next, it was analyzed herein the cell surface expression of CD44, CD54, MHC class I and PD-L1 after WIN 55,212-2 treatment by flow cytometry to evaluate its differentiation effect on oral tumor cells. Decreased expression of CD44, CD54, PD-L1 and MHC class I was detected in OSCCs after WIN 55,212-2 treatment when compared to untreated control. The effect was most pronounced with the highest concentration of WIN 55,212-2 (50 μM) (FIGS. 13, 18A). Similarly, the expression of CD44, CD54 and PD-L1 in OSCSCs was seen decreased after WIN 55,212-2 treatments. In contrast to OSCCs, MHC class I expression in OSCSCs remained unchanged after WIN 55,212-2 treatment (FIGS. 13 and 18A-18D). Significantly greater decrease of cell surface MHC class I expression after WIN 55,212-2 treatment was detected in OSCCs when compared to OSCSCs (FIG. 13).

Greater Decrease in Cell Proliferation and Higher Induction of Cell Death by WIN 55,212-2 in Stem-Like MP2 Tumor Cells than in Differentiated PL-12

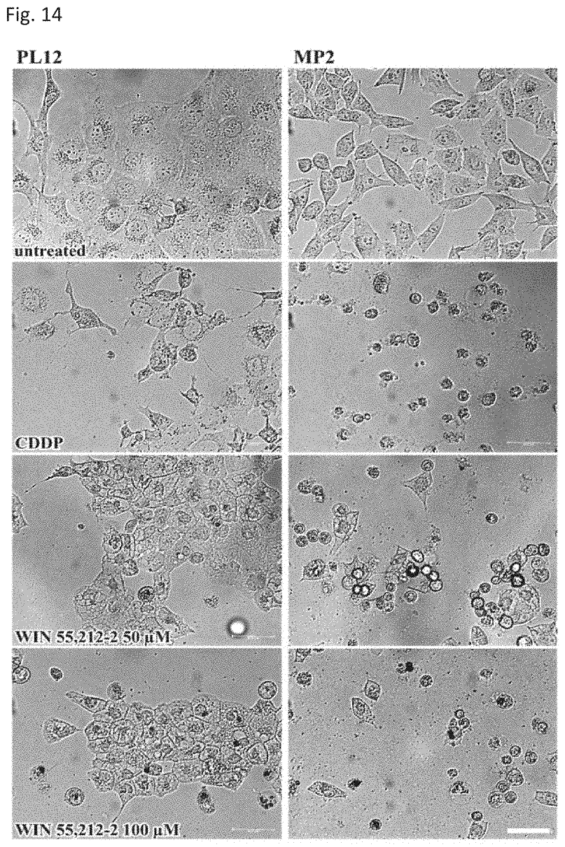

PL-12, well-differentiated pancreatic tumor cells, and MP2, poorly differentiated/stem like pancreatic tumor cells were treated with different concentrations of WIN 55,212-2 for 24 hours before their respective cell images were captured by microscopy. The results were compared to the treatment of the tumors with CDDP (FIG. 14). PL-12 maintained their morphology and remained viable after WIN 55,212-2 treatment, although some floating and non-viable cells were seen in the culture plates at the highest concentration of WIN 55,212-2 (FIG. 14). In contrast, MP2 had largely lost their shape and morphology, and had detached from the plates, and were sickly after treatment with WIN 55,212-2 (FIG. 14). In comparison to WIN 55,212-2, CDDP affected both PL-12 and MP2 morphologically (FIG. 14).

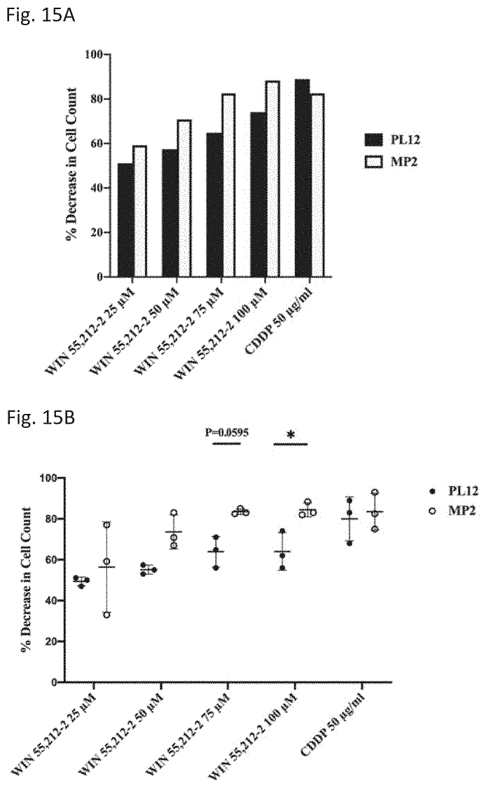

Viable cell numbers in the cell cultures were determined after treatment of tumors with different concentrations of WIN 55,212-2 using microscopy. Decreased numbers of viable cells were seen in both PL-12 and MP2 by 50-70% and 60-85%, respectively, in a dose-dependent manner. Therefore, there was a significantly greater decrease in cell numbers, at least in two concentrations, after WIN 55,212-2 treatment in MP2 when compared to PL-12 tumors. In addition, decreased numbers of MP2 by the highest concentrations of WIN 55,212-2 (75 and 100 μM) was similar to those seen in CDDP treated groups (FIGS. 15A, 15B).

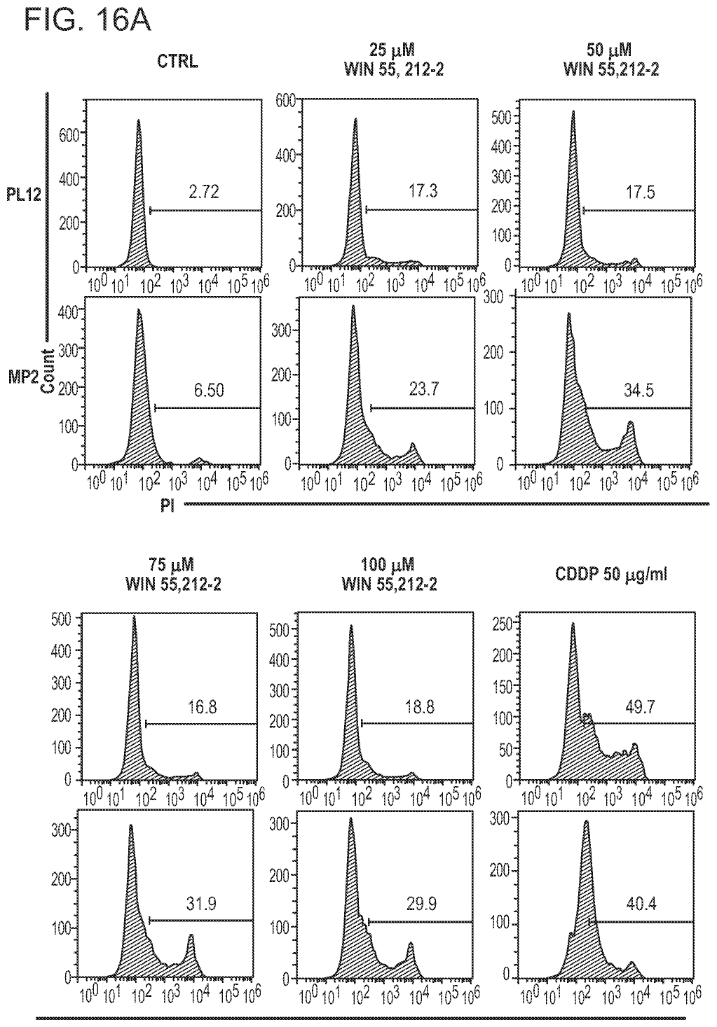

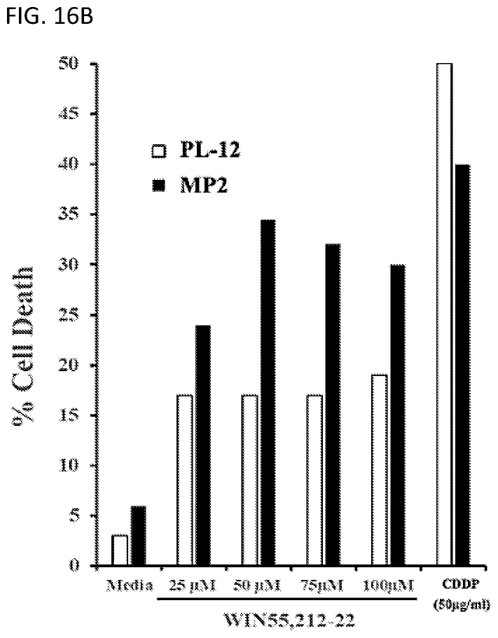

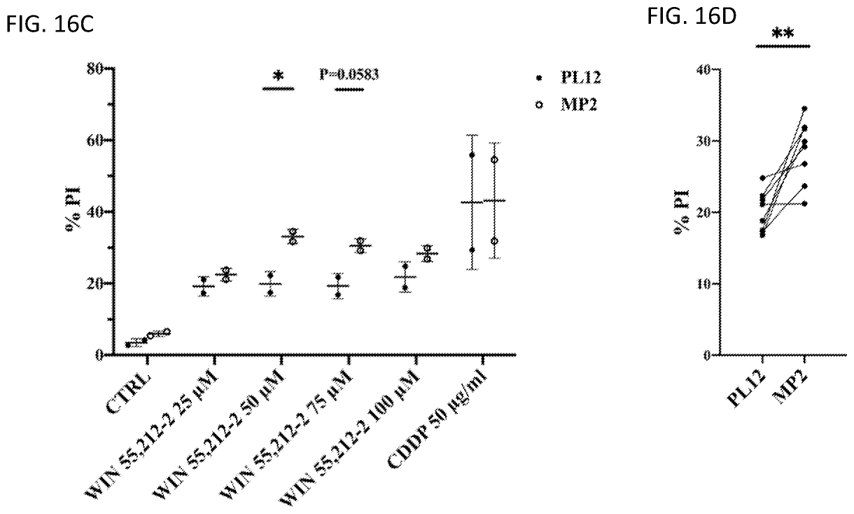

Next, it was measured herein the cell death using PI staining and flow cytometric analysis. Significantly higher percentages of dead cells were seen in poorly differentiated/stem-like MP2 (approximately 30%) when compared to differentiated PL-12 (up to 20%) after treatment with different concentrations of WIN 55,212-2 (25-100 μM). In contrast, cell death induced by CDDP was higher in PL-12 in the representative experiment (FIG. 16B) and similar in compiled data than those seen in MP2 tumors due to variability we see among the different experiments (FIG. 16C). In the paired compiled experiments PL12 has lower cell death when compared to MP2 tumor cells with different concentrations of WIN 55-212-2 (FIG. 16D).

Cell Surface Expressions of CD44, CD54, PD-L1 and MHC Class I were Down-Regulated in Pancreatic Tumor Cells after WIN 55,212-2 Treatment

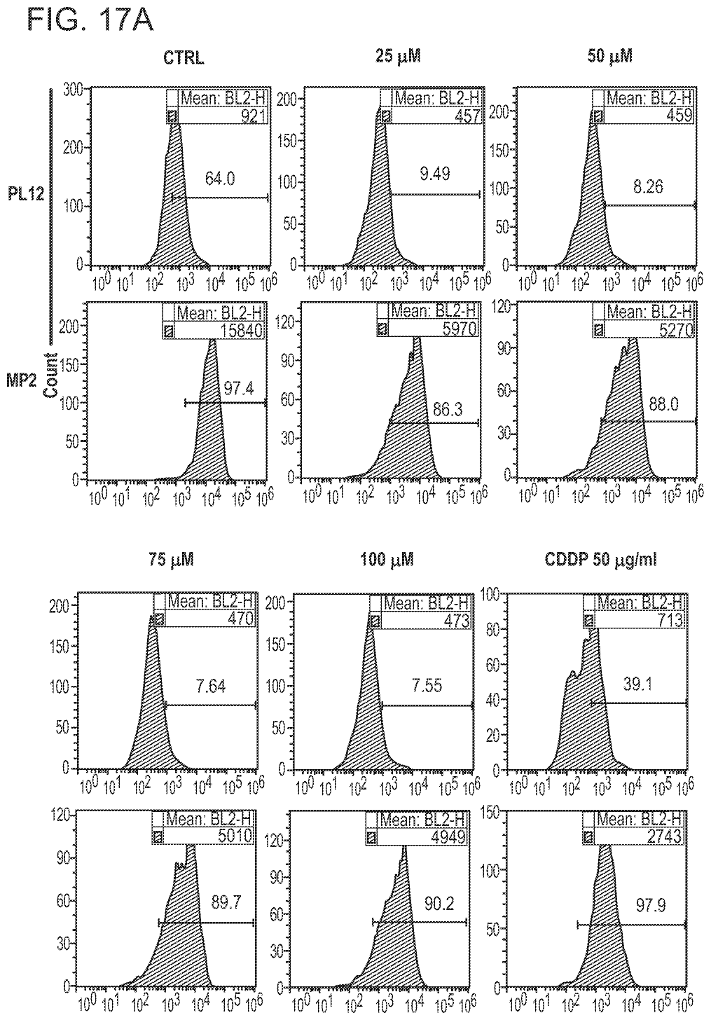

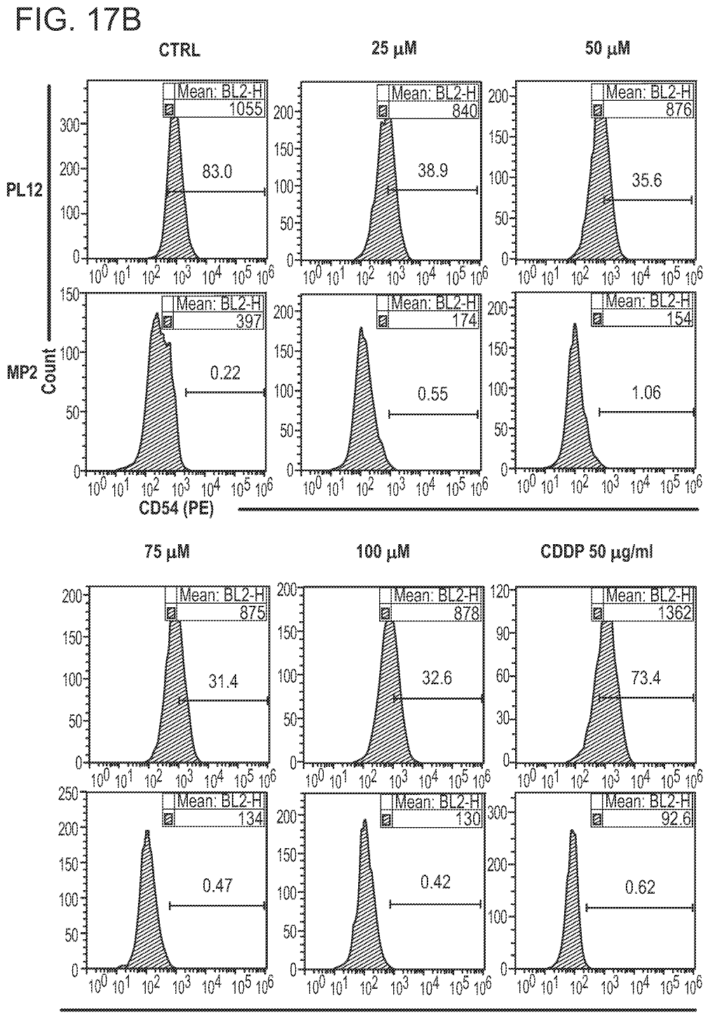

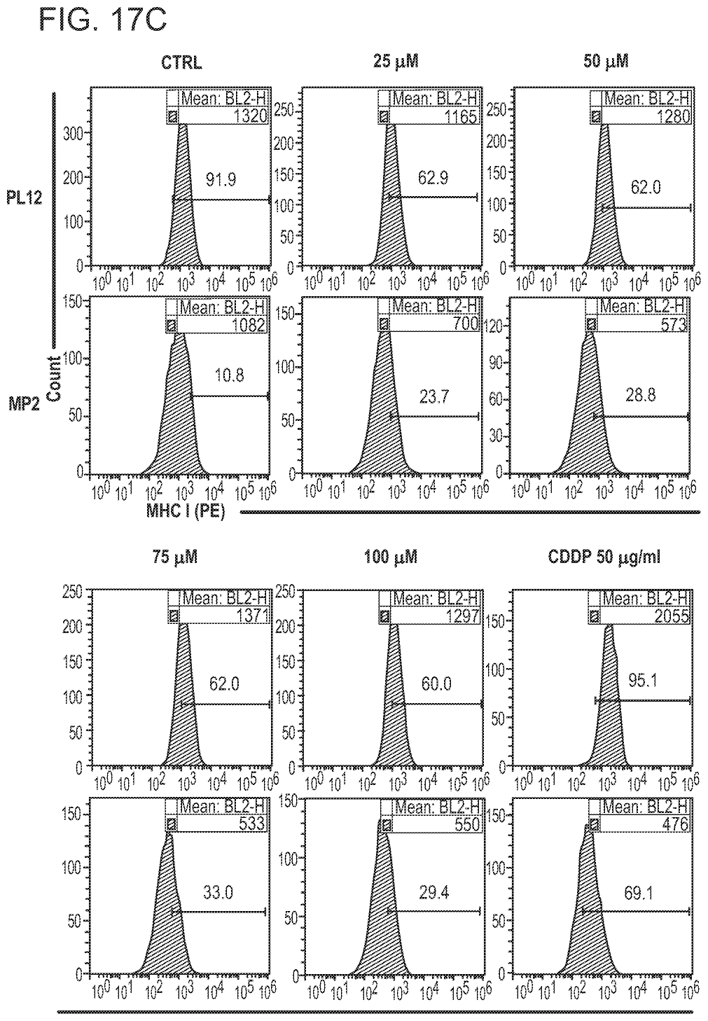

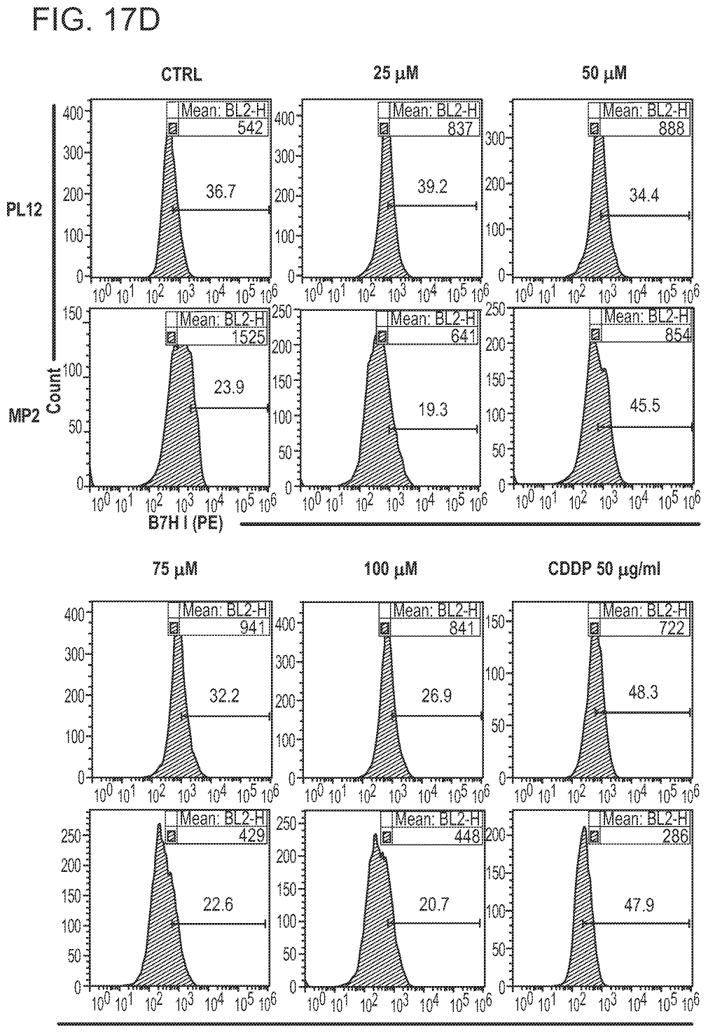

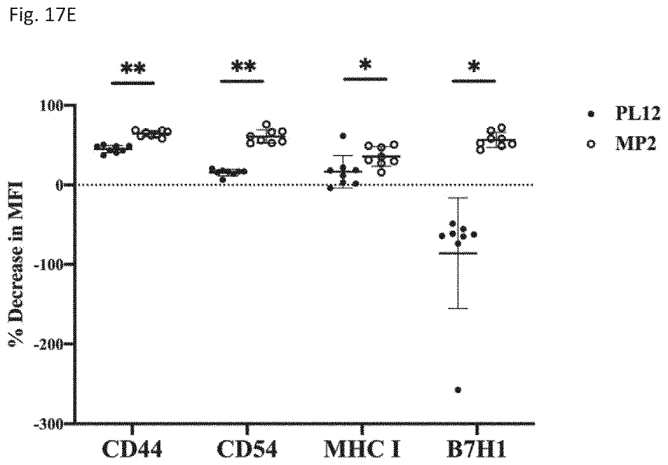

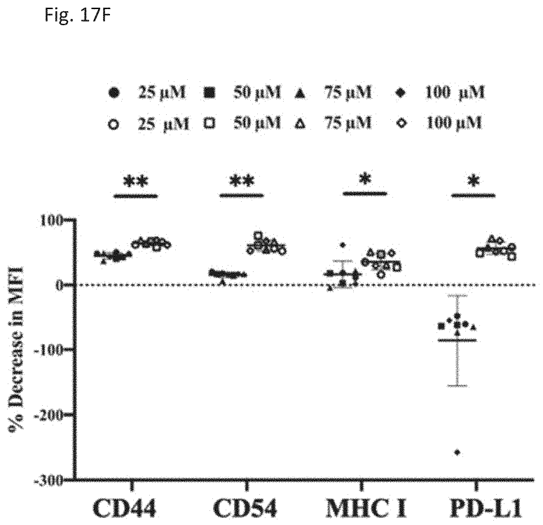

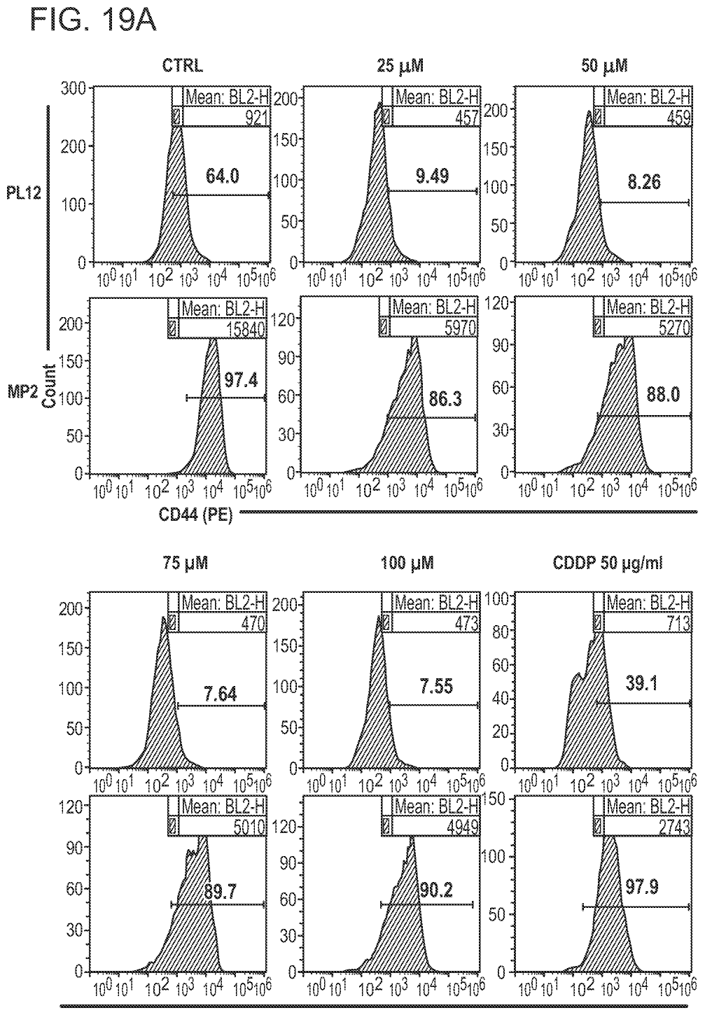

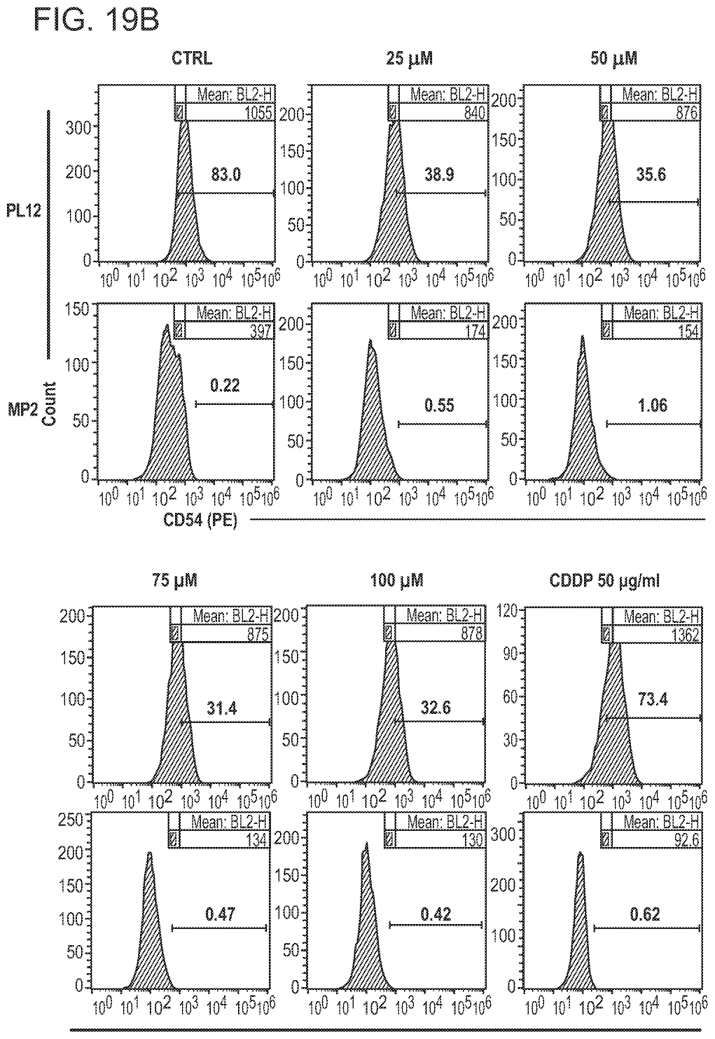

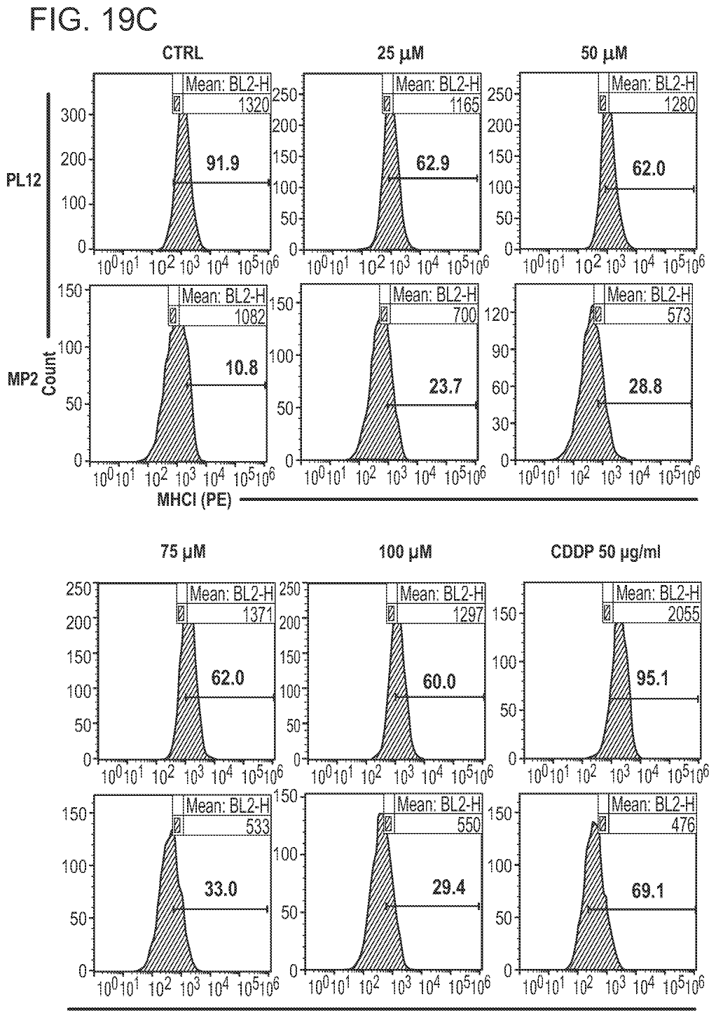

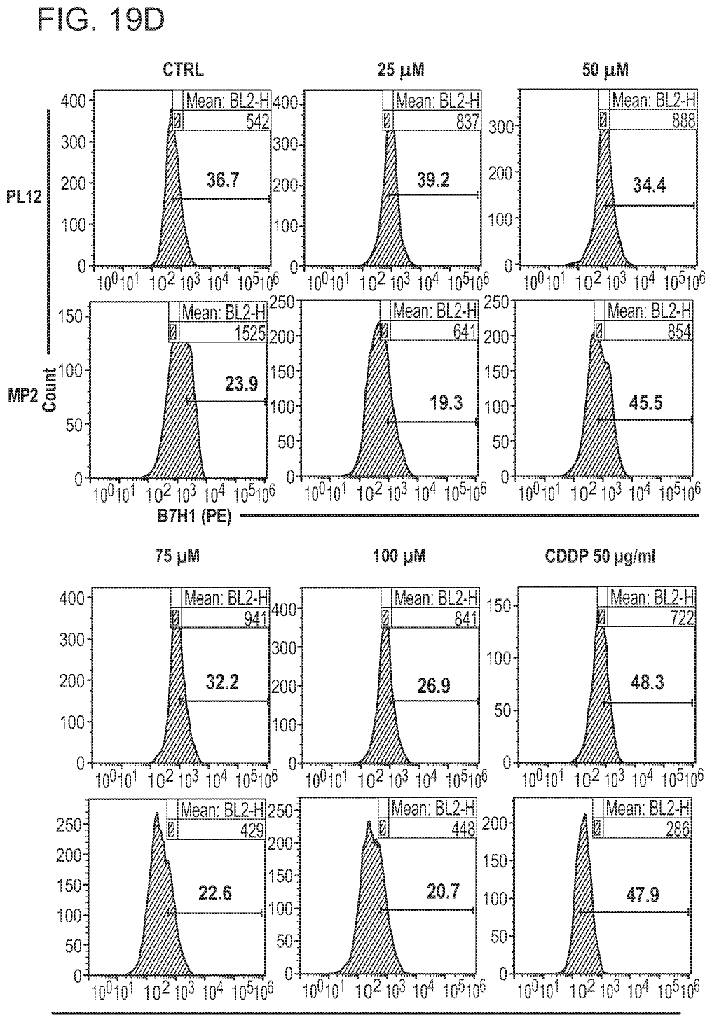

Cell surface expressions of CD44, CD54, PD-L1, and MHC class I were analyzed on PL-12 and MP2 after WIN 55,212-2 and CDDP treatments. Decreased cell surface expression of CD44 was seen in both PL-12 and MP2 tumor cells after 48 h of treatment with WIN 55,212-2. Similar to oral tumors, WIN 55,212-2 treatment decreased the expression of CD54 on both MP2 and PL-12. However, the expressions of PD-L1 were seen to be increased in PL-12 but decreased in MP2 after WIN 55,212-2 treatment. Akin to PD-L1, MHC class I expressions were found to be increased on PL-12 but decreased on MP2 tumor cells (FIGS. 17F and 19A-19D).

Taken together, WIN 55,212-2 treatment down-regulated expression of all cell surface receptors in differentiated PL-12 and poorly differentiated/stem-like MP2 tumor cells with the exception of PD-L1, in which expressions were up-regulated in PL-12 tumor cells after treatment. Also, the extent of decrease in all surface receptor expressions in MP2 tumor cells was significantly greater than those seen in PL-12 tumor cells after WIN 55,212-2 treatments (FIG. 17F).

### Expression of CB2R but not CB1R on Oral and Pancreatic Tumors

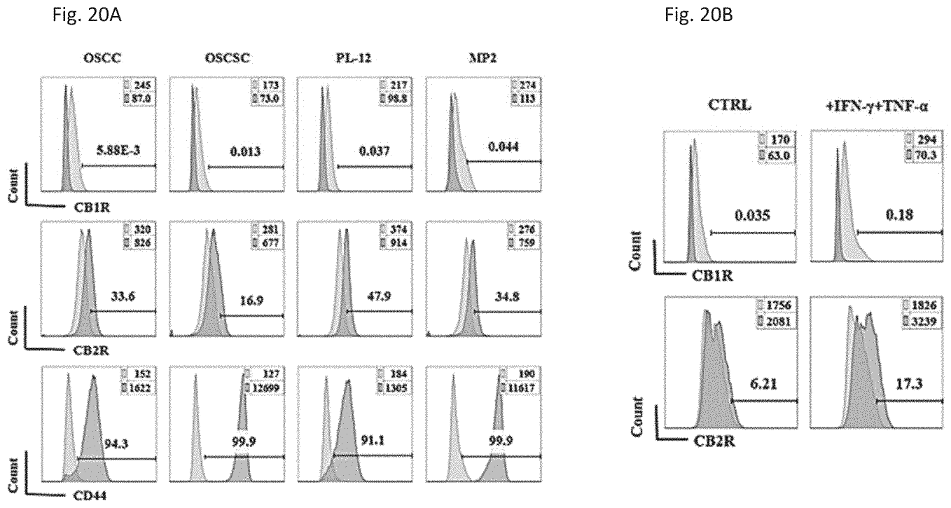

To determine whether there is a correlation between the expression of the CB1 and CB2 receptors and higher sensitivity to WIN 55,212-2 effect in tumor cells, it was assessed herein the levels of these receptors on the surface of both stem-like/poorly differentiated and well-differentiated oral and pancreatic tumors. The expression of CB1 receptors was not observed on any of the tumor cells tested (FIG. 20A). Increased expression of CB2 receptors was seen on both oral and pancreatic tumor cells, with well-differentiated tumors having higher expression than stem-like/poorly differentiated tumors both in oral and pancreatic tumor cells (FIG. 20A). As shown in FIG. 20A the levels of differentiation in OSCCs and PL-12 tumor cells is correlated with decreased expression of CD44 when compared to those expressed on the surface of OSCSCs and MP2 tumor cells. We next differentiated the stem-like/poorly differentiated tumor cells by the use of IFN-γ and TNF-α treatment as established previously, and assessed the levels of receptor expression. Treatment with IFN-γ and TNF-α increased CB2 receptor expression on stem-like/poorly differentiated tumors (FIG. 20B). Surprisingly, there was no correlation between CB2 receptor expression and increased cell death in stem-like/poorly differentiated tumors. Thus, (1) the lack of correlation between the CB2 receptor level and increased cell death in stem-like cancer cells, and (2) the lack of expression of the CB1 receptor on stem-like cancer cells, were unexpected since cannabinoid was thought to function exclusively via the CB1 and CB2 receptors.

### CONCLUSION

Thus far, it has been unclear the effect of cannabinoids on tumor cells in different stages of differentiation. It was determined herein the effect of cannabinoid on both well differentiated as well as poorly differentiated tumor cells. It is well established that CSCs and poorly differentiated cells are resistant to the effects of chemotherapy, whereas their well differentiated tumor cells are more susceptible. Indeed, the NK cells were the only cell type that was found to target CSCs/poorly differentiated tumor cells but not the well-differentiated tumor cells. Therefore, it is extremely important to find drugs or other factors that can target resistant CSCs/poorly differentiated tumor cells since these tumors seed the cancer and have metastatic potential, unlike the well-differentiated tumor cells. It is presented herein that synthetic cannabinoid WIN 55,212-2 target and kill CSCs/poorly differentiated tumor cells. Although WIN 55,212-2 can also target the well-differentiated tumor cells, its effect is more pronounced on CSCs/poorly differentiated tumor cells. This is different from those seen by chemotherapeutic drugs since these drugs have a greater ability to target well-differentiated tumor cells and in certain tumors they do not affect the course of CSCs/poorly differentiated tumor cells.

A number of oral and pancreatic tumor lines in different stages of differentiation have been previously characterized. By using four surface receptors of CD44, CD54, MHC class I and PD-L1, CSCs/poorly differentiated tumor cells have been differentiated from moderately differentiated tumor cells and well-differentiated oral and pancreatic tumor cells. CSCs/poorly differentiated tumor cells exhibited higher CD44 and lower or no expression of CD54, MHC class I and PD-L1 whereas well differentiated tumor cells expressed lower CD44 and higher expressions of CD54, MHC class I and PD-L1. These four surface antigens' expression have been used herein to differentiate the tumor cells and determine the effect of WIN 55,212-2 on tumor cells (e.g., oral and pancreatic tumor cells). It was observed herein that tumor cell surface expressions of these four receptors were greatly modulated/decreased on both oral and pancreatic tumors by WIN 55,212-2, likely due to the ability of this compound to block proliferation, induce cell death and/or modulate the surface receptors. The ability to decrease cell surface expression by WIN 55,212-2 were seen on both well-differentiated and CSCs/poorly differentiated tumor cells, even though, WIN 55,212-2 had greater ability to induce decrease in cell numbers and increase cell death in CSCs/poorly differentiated tumor cells when compared to well-differentiated tumor cells. This indicates that WIN 55,212-2 also sensitizes the well-differentiated tumor cells to NK cell mediated cytotoxicity since it decreased the levels of MHC class I expression which are known to inhibit the function of NK cells. In addition, PD-L1 expression is also decreased on MP2 tumor cells but not on PL-12 tumor cells, indicating that WIN 55,212-2 may have differential effects on the expression of PD-L1 on different tumor cell types. PD-L1 is known to inhibit the function of cytotoxic immune effectors by binding to PD-1. Therefore, by decreasing the levels of PD-L1 on stem-like/poorly differentiated tumor cells, WIN 55,212-2 can release the break on the immune cell function and increase their ability to lyse tumors, however, by increasing PD-L1 on PL-12 tumors it may induce the opposite effect, in which it may block the immune function through increased binding to PD-1. However, it was shown that, cannabinoid use with anti-PD-1 agent nivolumab decreased the response rate to therapy in patients with advanced malignancies (Taha et al. (2019) Oncologist 24(4):549-554), indicating that WIN 55,212-2 has differential effect on tumors depending on the tumor type and their differentiation status.

The present study demonstrated that the effect of WIN 55,212-2 on CSCs/poorly differentiated tumor cells as compared to well-differentiated tumor cells could not have been foreseen by the level of cannabinoid receptors. The present study demonstrated a lack of increased or differential expression of the CB1 receptor on stem-like tumor cells or their differentiated counterparts (FIGS. 20A, 20B). In addition, it was demonstrated herein that the CB2 receptor expression was higher on differentiated OSCCs and PL-12 tumor cells compared to stem-like OSCSC and MP2 tumor cells (FIG. 20A). Similarly, when OSCSCs were differentiated by using the combination of IFN-γ and TNF-α, it was observed herein an increase in CB2 receptor expression (FIG. 20B). Thus, it is surprising that despite the lower level of cannabinoid receptors, WIN 55,212-2 shows preferential killing of CSCs/poorly differentiated tumor cells than well-differentiated tumor cells. It is possible that WIN 55,212-2 activates the CB1 or CB2 receptors differentially on CSCs/poorly differentiated tumor cells vs. those on well-differentiated tumor cells. Such unexpected finding indicates that synthetic cannabinoid WIN 55,212-2 provides an ideal therapy for treating patients with aggressive and metastatic tumors driven by CSCs/poorly differentiated tumors.

### REFERENCES

1. Velasco, G., C. Sanchez, and M. Guzman, Anticancer mechanisms of cannabinoids. Curr Oncol, 2016. 23(2): p. S23-32.
2. Fraguas-Sanchez, A. I., C. Martin-Sabroso, and A. I. Torres-Suarez, Insights into the effects of the endocannabinoid system in cancer: a review. Br J Pharmacol, 2018. 175(13): p. 2566-2580.
3. Qamri, Z., et al., Synthetic cannabinoid receptor agonists inhibit tumor growth and metastasis of breast cancer. Mol Cancer Ther, 2009. 8(11): p. 3117-29.
4. Preet, A., et al., Cannabinoid receptors, CB1 and CB2, as novel targets for inhibition of non-small cell lung cancer growth and metastasis. Cancer Prev Res (Phila), 2011. 4(1): p. 65-75.
5. Moreno, E., et al., The Endocannabinoid System as a Target in Cancer Diseases: Are We There Yet? Front Pharmacol, 2019. 10: p. 339.
6. Silva, A. G., et al., WIN55,212-2 induces caspase-independent apoptosis on human glioblastoma cells by regulating HSP70, p53 and Cathepsin D. Toxicol In Vitro, 2019. 57: p. 233-243.
7. Khan, M. I., et al., Involvement of the CB2 cannabinoid receptor in cell growth inhibition and G0/G1 cell cycle arrest via the cannabinoid agonist WIN 55,212-2 in renal cell carcinoma. BMC Cancer, 2018. 18(1): p. 583.
8. Xu, D., et al., Cannabinoid WIN55, 212-2 induces cell cycle arrest and inhibits the proliferation and migration of human BEL7402 hepatocellular carcinoma cells. Mol Med Rep, 2015. 12(6): p. 7963-70.
9. Zhang, G., et al., Inhibition of autophagy and enhancement of endoplasmic reticulum stress increase sensitivity of osteosarcoma Saos-2 cells to cannabinoid receptor agonist WIN55,212-2. Cell Biochem Funct, 2016. 34(5): p. 351-8.
10. Casanova, M. L., et al., Inhibition of skin tumor growth and angiogenesis in vivo by activation of cannabinoid receptors. J Clin Invest, 2003. 111(1): p. 43-50.
11. Sarfaraz, S., et al., Cannabinoid receptor as a novel target for the treatment of prostate cancer. Cancer Res, 2005. 65(5): p. 1635-41.
12. Luca, T., et al., The CB1/CB2 receptor agonist WIN-55,212-2 reduces viability of human Kaposi's sarcoma cells in vitro. Eur J Pharmacol, 2009. 616(1-3): p. 16-21.
13. Flygare, J., et al., Cannabinoid receptor ligands mediate growth inhibition and cell death in mantle cell lymphoma. FEBS Lett, 2005. 579(30): p. 6885-9.
14. Blazquez, C., et al., Cannabinoid receptors as novel targets for the treatment of melanoma. FASEB J, 2006. 20(14): p. 2633-5.
15. Emery, S. M., et al., Combined antiproliferative effects of the aminoalkylindole WIN55,212-2 and radiation in breast cancer cells. J Pharmacol Exp Ther, 2014. 348(2): p. 293-302.
16. Greish, K., et al., Synthetic cannabinoids nano-micelles for the management of triple negative breast cancer. J Control Release, 2018. 291: p. 184-195.
17. Xian, S., et al., The Use of Styrene Maleic Acid Nanomicelles Encapsulating the Synthetic Cannabinoid Analog WIN55,212-2 for the Treatment of Cancer. Anticancer Res, 2015. 35(9): p. 4707-12.
18. Heppner, G. H. and B. E. Miller, Tumor heterogeneity: biological implications and therapeutic consequences. Cancer Metastasis Rev, 1983. 2(1): p. 5-23.
19. Tseng, H. C., N. Cacalano, and A. Jewett, Split anergized Natural Killer cells halt inflammation by inducing stem cell differentiation, resistance to NK cell cytotoxicity and prevention of cytokine and chemokine secretion. Oncotarget, 2015. 6(11): p. 8947-59.
20. Tseng, H. C., et al., Increased lysis of stem cells but not their differentiated cells by natural killer cells; de-differentiation or reprogramming activates NK cells. PLoS One, 2010. 5(7): p. e11590.
21. Tseng, H. C., et al., Induction of Split Anergy Conditions Natural Killer Cells to Promote Differentiation of Stem Cells through Cell-Cell Contact and Secreted Factors. Front Immunol, 2014. 5: p. 269.
22. Tseng, H. C., et al., Differential Targeting of Stem Cells and Differentiated Glioblastomas by NK Cells. J Cancer, 2015. 6(9): p. 866-76.
23. Kaur, K., et al., Probiotic-Treated Super-Charged NK Cells Efficiently Clear Poorly Differentiated Pancreatic Tumors in Hu-BLT Mice. Cancers (Basel), 2019. 12(1).
24. Jewett, A. and B. Bonavida, Target-induced inactivation and cell death by apoptosis in a subset of human NK cells. J Immunol, 1996. 156(3): p. 907-15.
25. Jewett, A., et al., Cytokine dependent inverse regulation of CD54 (ICAM1) and major histocompatibility complex class I antigens by nuclear factor kappaB in HEp2 tumor cell line: effect on the function of natural killer cells. Hum Immunol, 2003. 64(5): p. 505-20.
26. Kozlowska, A. K., et al., Differentiation by NK cells is a prerequisite for effective targeting of cancer stem cells/poorly differentiated tumors by chemopreventive and chemotherapeutic drugs. J Cancer, 2017. 8(4): p. 537-554.
27. Kaur, K., et al., Super-charged NK cells inhibit growth and progression of stem-like/poorly differentiated oral tumors in vivo in humanized BLT mice; effect on tumor differentiation and response to chemotherapeutic drugs. Oncoimmunology, 2018. 7(5): p. e1426518.
28. Tseng, H. C., N. Cacalano, and A. Jewett, Split anergized Natural Killer cells halt inflammation by inducing stem cell differentiation, resistance to NK cell cytotoxicity and prevention of cytokine and chemokine secretion. Oncotarget, 2015.
29. Jewett, A., et al., Multiple Defects of Natural Killer Cells in Cancer Patients: Anarchy, Dysregulated Systemic Immunity, and Immunosuppression in Metastatic Cancer. Crit Rev Immunol, 2020. 40(2): p. 93-133.
30. Kaur, K., et al., Novel Strategy to Expand Super-Charged NK Cells with Significant Potential to Lyse and Differentiate Cancer Stem Cells: Differences in NK Expansion and Function between Healthy and Cancer Patients. Front Immunol, 2017. 8: p. 297.
31. Jewett, A., et al., NK cells shape pancreatic and oral tumor microenvironments; role in inhibition of tumor growth and metastasis. Semin Cancer Biol, 2018. 53: p. 178-188.
32. Kaur, K., et al., Natural killer cells target and differentiate cancer stem-like cells/undifferentiated tumors: strategies to optimize their growth and expansion for effective cancer immunotherapy. Curr Opin Immunol, 2018. 51: p. 170-180.
33. Jewett, A., et al., Natural Killer Cells: Diverse Functions in Tumor Immunity and Defects in Pre-neoplastic and Neoplastic Stages of Tumorigenesis. Mol Ther Oncolytics, 2020. 16: p. 41-52.
34. Concha-Benavente, F., et al., PD-L1 Mediates Dysfunction in Activated PD-1(+) NK Cells in Head and Neck Cancer Patients. Cancer Immunol Res, 2018. 6(12): p. 1548-1560.
35. Xu, L., et al., Adipocytes affect castration-resistant prostate cancer cells to develop the resistance to cytotoxic action of NK cells with alterations of PD-L1/NKG2D ligand levels in tumor cells. Prostate, 2018. 78(5): p. 353-364.
36. Taha, T., et al., Cannabis Impacts Tumor Response Rate to Nivolumab in Patients with Advanced Malignancies. Oncologist, 2019. 24(4): p. 549-554.
37. Xu, X., et al., Overexpression of cannabinoid receptors CB1 and CB2 correlates with improved prognosis of patients with hepatocellular carcinoma. Cancer Genet Cytogenet, 2006. 171(1): p. 31-8.
38. Carracedo, A., et al., Cannabinoids induce apoptosis of pancreatic tumor cells via endoplasmic reticulum stress-related genes. Cancer Res, 2006. 66(13): p. 6748-55.
39. Felder, C. C., et al., Comparison of the pharmacology and signal transduction of the human cannabinoid CB1 and CB2 receptors. Mol Pharmacol, 1995. 48(3): p. 443-50.
40. Shire, D., et al., Molecular cloning, expression and function of the murine CB2 peripheral cannabinoid receptor. Biochim Biophys Acta, 1996. 1307(2): p. 132-6.
41. Cianchi, F., et al., Cannabinoid receptor activation induces apoptosis through tumor necrosis factor alpha-mediated ceramide de novo synthesis in colon cancer cells. Clin Cancer Res, 2008. 14(23): p. 7691-700.
42. Wright, K., et al., Differential expression of cannabinoid receptors in the human colon: cannabinoids promote epithelial wound healing. Gastroenterology, 2005. 129(2): p. 437-53.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of preventing or treating a cancer in a subject, comprising administering to the subject a composition comprising a cannabinoid or a pharmaceutically acceptable salt thereof.

2. The method of claim 1, wherein the cancer comprises:
(a) cancer stem cells, poorly differentiated cancer cells, and/or undifferentiated cancer cells; and/or
(b) cancer cells with:
(i) an increased level of CD44, CD26, CD166, CD326, CD338, and/or CD133;
(ii) a decreased level of CD54, PD-L1, and/or MHC class I on the cancer cell surface compared to differentiated cells; and/or
(iii) susceptibility to NK cell-mediated cytotoxicity.

3. (canceled)

4. The method of claim 1, wherein the cannabinoid is:
(a) a cannabinoid receptor agonist or antagonist;
(b) a cannabinoid receptor agonist of CB1R and/or CB2R;
(c) synthetic or naturally occurring;
(d) in a pharmaceutical composition; and/or
(e) WIN 55,212-2.

5-8. (canceled)

9. The method of claim 1, wherein the subject is treated conjointly with at least one cancer therapy, optionally wherein the subject is treated with at least one cancer therapy before, after, or concurrently with the composition comprising a cannabinoid.

10. The method of claim 9, wherein the at least one cancer therapy is:
(a) selected from a surgery, radiation therapy, chemotherapy, immunotherapy, and a combination thereof;
(b) chemotherapy comprising CDDP;
(c) immunotherapy that inhibits immune checkpoint;
(d) immunotherapy that inhibits immune checkpoint, wherein the immune checkpoint is
(i) selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR; or
(ii) PD-1 or PD-L1; or
(e) immunotherapy comprising an NK cell therapy.

11-16. (canceled)

17. The method of claim 1, wherein the cancer is:
(a) a solid or a hematological cancer;
(b) a metastatic cancer;
(c) selected from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and glioblastoma; or
(d) selected from a pancreatic cancer and an oral cancer.

18-20. (canceled)

21. The method of claim 1, wherein:
(a) the subject is treated with a composition comprising WIN 55,212-2, chemotherapy, and an immunotherapy, wherein the immunotherapy inhibits PD-1 or PD-L1, optionally wherein the subject is afflicted with a pancreatic cancer;
(b) the composition comprising a cannabinoid is administered by inhalation, oral administration, parenteral administration, sublingual administration, topical administration, intravenous administration, intratumoral administration, intramuscular administration, or subcutaneous administration; and/or
(c) the method decreases the amount of at least one cell surface antigen on a cancer cell, wherein the at least one cell surface antigen is selected from CD44, CD26, CD166, CD326, CD338, CD133, CD54, MEW class I, and PD-L1.

22. The method of claim 1, wherein the subject is a mammal.

23. (canceled)

24. A method of inhibiting the proliferation of a cancer cell, comprising contacting the cancer cell with a composition comprising a cannabinoid or a pharmaceutically acceptable salt thereof.

25. The method of claim 24, wherein:
(a) the cancer cell is a cancer stem cell, a poorly differentiated cancer cell, and/or an undifferentiated cancer cell; or
(b) the cancer cell has:
(i) an increased level of CD44, CD26, CD166, CD326, CD338, and/or CD133;
(ii) a decreased level of CD54, PD-L1, and/or MHC class I on the cancer cell surface compared to differentiated cells; and/or
(iii) susceptibility to NK cell-mediated cytotoxicity.

26. (canceled)

27. The method of claim 24, wherein the cannabinoid is:
(a) a cannabinoid receptor agonist or antagonist;
(b) a cannabinoid receptor agonist of CB1R and/or CB2R;
(c) synthetic or naturally occurring;
(d) in a pharmaceutical composition; and/or
(e) WIN 55,212-2.

28-31. (canceled)

32. The method of claim 24, wherein the cancer cell is contacted conjointly with at least one cancer therapy, optionally wherein the cancer cell is contacted with at least one cancer therapy before, after, or concurrently with the composition comprising a cannabinoid.

33. The method of claim 32, wherein the at least one cancer therapy is:
(a) selected from a surgery, radiation therapy, chemotherapy, immunotherapy, and a combination thereof;
(b) chemotherapy comprising CDDP;
(c) immunotherapy that inhibits immune checkpoint;
(d) immunotherapy that inhibits immune checkpoint, wherein the immune checkpoint is
(i) selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR, or
(ii) PD-1 or PD-L1; and/or
(e) immunotherapy comprising an NK cell therapy.

34-39. (canceled)

40. The method of claim 24, wherein the cancer cell is of:
(a) a solid or a hematological cancer;
(b) a metastatic cancer;
(c) a cancer selected from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and glioblastoma; or
(d) a cancer selected from a pancreatic cancer and an oral cancer.

41-43. (canceled)

44. The method of claim 24, wherein:
(a) the cancer cell is contacted with a composition comprising WIN 55,212-2, chemotherapy, and an immunotherapy, wherein the immunotherapy inhibits PD-1 or PD-L1, optionally wherein the cancer cell is a pancreatic cancer cell;
(b) the cancer cell is contacted with the composition in vitro, ex vivo, or in vivo, and/or
(c) the method decreases the amount of at least one cell surface antigen on the cancer cell, wherein the at least one cell surface antigen is selected from CD44, CD26, CD166, CD326, CD338, CD133, CD54, MHC class I, and PD-L1.

45. The method of claim 24, wherein the cancer cell is of a mammal.

46. (canceled)

47. A method of determining whether a subject afflicted with a cancer would benefit from a composition comprising a cannabinoid or a pharmaceutically acceptable salt thereof, the method comprising:
a) determining the amount of at least one biomarker selected from CD44, CD26, CD166, CD326, CD338, CD133, CD54, PD-L1, and MHC class I in a subject sample comprising a cancer cell;
b) determining the amount of the at least one biomarker in a control (e.g., differentiated cells, e.g., differentiated cancer cells or differentiated non-cancerous cells) (e.g., preferably of the same cell type); and
c) comparing the amount of the at least one biomarker detected in steps a) and b);
wherein a significantly higher amount of CD44, CD26, CD166, CD326, CD338, and/or CD133; and/or a significantly lower amount of CD54, PD-L1, and/or MHC class I in the subject sample indicates that the subject would benefit from the composition comprising a cannabinoid or a pharmaceutically acceptable salt thereof.

48. The method of claim 47, further comprising recommending, prescribing, or administering
a) the composition comprising the cannabinoid or a pharmaceutically acceptable salt thereof to the subject, if the subject is determined to benefit from the composition comprising a cannabinoid or a pharmaceutically acceptable salt thereof; or
b) a therapy other than the composition comprising the cannabinoid or a pharmaceutically acceptable salt thereof to the subject, if the subject is determined not to benefit from the composition comprising a cannabinoid or a pharmaceutically acceptable salt thereof.

49-50. (canceled)

51. The method of claim 47, wherein the cannabinoid is:
(a) a cannabinoid receptor agonist or antagonist,
(b) a cannabinoid receptor agonist of CB1R and/or CB2R;
(c) synthetic or naturally occurring;
(d) in a pharmaceutical composition; and/or
(e) WIN 55,212-2.

52. (canceled)

53. The method of claim 47, wherein the cancer or cancer cell is:
(a) of a solid or a hematological cancer;
(b) a metastatic cancer;
(c) selected from multiple myeloma, prostate cancer, stomach cancer, bladder cancer, esophageal cancer, cervical cancer, liver cancer, kidney cancer, bone cancer, brain cancer, leukemia, head and neck cancer, oral cancer, pancreatic cancer, lung cancer, colon cancer, melanoma, breast cancer, ovarian cancer, and glioblastoma; or
(d) selected from a pancreatic cancer and an oral cancer.

54-55. (canceled)

56. The method of claim 47, wherein the subject is a mammal.
